(19) United States
(12) Patent Application Publication
(10) Pub. No.: US 2026/0014083 A1
(43) Pub. Date: Jan. 15, 2026

(54) Orally Administrable Pharmaceutical Dosage Form Comprising Lanthanum and Its Use in a Method Of Treatment of Hyperoxaluria

(71) Applicant: AMGMT, Berchem - Antwerpen (BE)

(72) Inventor: Marc De Broe, Lokeren (BE)

(21) Appl. No.: 18/880,447

(22) PCT Filed: Jun. 30, 2023
(86) PCT No.: PCT/EP2023/067969

§ 371 Date: Dec. 31, 2024

(30) Foreign Application Priority Data
Jul. 1, 2022 (EP) 22182615.9

(51) Int. Cl. A61K 9/48; A61K 9/00; A61K 33/244; A61P 13/02; A61P 13/12

(52) U.S. Cl. CPC A61K 9/4891; A61K 9/4808; A61K 9/4816; A61K 33/244; A61P 13/02; A61P 13/12; A61K 9/0053

(57) ABSTRACT

The invention concerns an orally administrable pharmaceutical dosage form configured for targeted delivery to the distal part of the gastrointestinal tract of a human subject, wherein the pharmaceutical dosage form comprises lanthanum or a pharmaceutically acceptable salt or oxide thereof. The invention further relates to the pharmaceutical dosage form for use in the treatment of hyperoxaluria in a human subject, optionally wherein the hyperoxaluria is primary hyperoxaluria or secondary hyperoxaluria; and/or for use in the treatment of one or more of nephrolithiasis, nephrocalcinosis, or oxalosis in a human subject.

### FIELD OF THE INVENTION

The invention is broadly in the field of medicine, more precisely in the field of nephrology and urology. In particular, the invention concerns an orally administrable pharmaceutical dosage form and it use for treatment of primary and secondary hyperoxaluria in a subject and/or for treatment of one or more of nephrolithiasis, nephrocalcinosis, or oxalosis in a subject.

### BACKGROUND OF THE INVENTION

Hyperoxaluria is a metabolic disorder characterized by significantly elevated oxalate levels in the urine, due to either overproduction of oxalate by the liver from a genetic defect, called primary hyperoxaluria, or from the excess absorption of oxalate into the gastrointestinal (GI) tract followed by excretion in the urine, called secondary hyperoxaluria. Secondary hyperoxaluria is further characterized either as dietary, resulting from excess intake of oxalate or oxalate precursors; enteric, resulting from a chronic gastrointestinal disorder associated with malabsorption, such as Crohn's disease (ileitis terminalis) or short bowel syndrome as a result of bariatric surgery, which predisposes patients to excess oxalate absorption; or idiopathic, meaning the underlying cause is unknown. Kidney stones, typically the first sign of hyperoxaluria, are often painful and may require interventional procedures. Severe hyperoxaluria in settings of enteric and primary hyperoxaluria may also lead to kidney damage (nephrocalcinosis), chronic kidney disease and end-stage renal disease, which may lead to death.

Nephrolithiasis, also known as kidney stone disease or urolithiasis, is one of the most common renal urological diseases. Recent estimates place the prevalence in the United States (US) population at 10.6% for men and 7.1% for women (Ziemba et al., 2017, Inv. Clin. Urol., 58, 299). Comparable prevalence is observed in Europe, and Japan. Eighty percent of all renal stones are calcium oxalate (CaOx) stones. Over the last 10 years, no major breakthrough of new potential medications in the treatment of nephrolithiasis or nephrocalcinosis was noted.

Lanthanum carbonate, an intestinal phosphate binder (e.g., Fosrenol, Shire Pharmaceuticals, Dublin, Ireland), has been shown in an oxalate loading (high doses) model in rat to decrease the oxaluria and the CaOx crystalluria. However, this finding went along with a striking increase of the phosphaturia and a minor increase of the calciuria (Robijn et al., 2013, J. Urol., 189(5):1960-1966). The use of lanthanum carbonate as such in patients with normal or slightly decreased renal function is associated with development of phosphate depletion over time. Phosphate depletion has been shown to result in increased bone resorption, thereby leading to severe side effects such as osteomalacia, bone fractures and several serious extraskeletal complications (Yvey et al., 1978, Adv Exp Med Biol, 193,373-380).

Recently, Allena Pharmaceuticals reported positive results from a clinical trial with reloxaliase, an oral enzyme therapeutic, in patients with enteric hyperoxaluria (EH), a more severe subset of secondary hyperoxaluria. The study achieved its primary endpoint, with a mean reduction of 22.6% in average 24-hour urinary oxalate excretion measured during weeks 1-4 among patients treated with reloxaliase, compared to 9.7% in the placebo group (least square mean treatment difference of −14.3%, p=0.004).

Further, Alnylam Pharmaceuticals announced positive phase 3 study results for lumasiran (brand name Oxlumo). This RNAi drug, is a double-stranded siRNA linked to n-acetyl-galactosamine to promote liver uptake for the targeting of glycolate, and is targeting glycolate oxidase involved in development of the high amounts of oxalate in primary hyperoxaluria type 1 treatment. However, use of lumasiran is limited to treatment of type 1 primary hyperoxaluria, the response is not optimal, and the cost of this treatment is planned to be particularly high.

Therefore, there remains a need in the art for further and/or improved treatment options for the treatment including prevention of primary and secondary hyperoxaluria in patients, and for the treatment including prevention of one or more of nephrolithiasis, nephrocalcinosis, or oxalosis in patients.

### SUMMARY OF THE INVENTION

As illustrated in FIG. 1, the present inventors have found the optimal site in the gut, namely the distal part of the gastrointestinal tract including the ileum and colon, where release of lanthanum should take place and chelation of oxalate should start to get the combined effect of reduction of hyperoxaluria without phosphate depletion, thereby addressing one or more of the above-mentioned problems in the art.

Accordingly, a first aspect of the invention relates to an orally administrable pharmaceutical dosage form configured for targeted delivery to the distal part of the gastrointestinal tract of a subject, wherein the pharmaceutical dosage form comprises lanthanum or a pharmaceutically acceptable salt or oxide thereof.

Preferably, a first aspect of the invention relates to an orally administrable pharmaceutical dosage form comprising lanthanum or a pharmaceutically acceptable salt or oxide thereof, wherein the pharmaceutical dosage form is configured for targeted delivery of the lanthanum or pharmaceutically acceptable salt or oxide thereof to one or more of the ileum, cecum, or colon of a human subject.

As illustrated in the example section, the present pharmaceutical dosage form allows release of lanthanum and chelation of oxalate only in the distal part of the gastrointestinal tract, such as in one or more of the ileum, cecum, or colon, thereby decreasing oxalate absorption in the distal gastrointestinal tract and decreasing urinary oxalate concentration. In addition, the present pharmaceutical dosage form allows to redirect oxalate from the serum to the intestinal lumen by secretion. These effects are combined with a calcium channel blocking effect of lanthanum in the distal part of the gastrointestinal tract resulting in a substantial decrease of urinary oxalate and normal or slightly decreased urinary calcium concentrations, thereby arresting oxalate calcium crystal formation and decreasing nephrolithiasis. Moreover, the present pharmaceutical dosage form impedes interactions of lanthanum with phosphate salts in the proximal gastrointestinal tract, i.e. duodenum and jejunum, thereby allowing phosphate reabsorption and preventing phosphate depletion.

Hence, a further aspect relates to the pharmaceutical dosage form as taught herein for use in the treatment (including prevention) of hyperoxaluria in a subject, optionally wherein the hyperoxaluria is primary hyperoxaluria or secondary hyperoxaluria. Preferably, the subject is a human subject.

A yet further aspect relates to the pharmaceutical dosage form as taught herein for use in the treatment (including prevention) of one or more of nephrolithiasis, nephrocalcinosis, or oxalosis in a subject, such as one or more of nephrolithiasis, nephrocalcinosis, or oxalosis caused by or related to calcium oxalate formation in a subject. Preferably, the subject is a human subject.

Related aspects provide a method of treating one or more of hyperoxaluria, nephrolithiasis, nephrocalcinosis, or oxalosis in a human subject in need of such a treatment, comprising administering a therapeutically effective amount of a pharmaceutical dosage form as defined herein to the subject. Preferably, the hyperoxaluria is primary hyperoxaluria or secondary hyperoxaluria.

Based on a proof of concept study, excellent results were obtained in three patients with primary oxaluria and in one patient with secondary hyperoxaluria. After treatment of the subjects, in particular human subjects, with the present pharmaceutical dosage form, the present inventors found:
specific release of lanthanum in the ileum and early colon;
optimal gastrointestinal chelation of oxalate in the ileum and colon resulting in substantial decrease in oxalate urinary concentration in addition to normal or even slightly decreased calciuria, e.g. oxalate urinary concentration below 0.50 mmol/L (44 mg/L) which is considered as the oxalate concentration without crystal formation provided the calcium concentration is below 4 mmol/L (160 mg/L), which was the case for three out of four primary hyperoxaluria patients and for the secondary hyperoxaluria patient;
normal and stable urinary phosphate concentrations and normal tubular phosphate reabsorption resulting in a normal phosphate balance during the whole treatment period;
the same or even slightly decreased calcium concentration in the urine during the whole treatment period; and
absence of an increase in calciuria likely due to calcium channel blocking of lanthanum in the gastrointestinal tract.

The present inventors have found that the present pharmaceutical dosage form arrests calcium oxalate crystal formation in the tubular fluid of the kidney and urine without causing phosphate depletion. Since calcium oxalate crystals are the first critical step in the formation of renal calculi, eventually causing nephrolithiasis, the present pharmaceutical dosage form advantageously allows to reduce hyperoxaluria and even avoid nephrolithiasis, nephrocalcinosis, and oxalosis, in particular related to calcium oxalate deposition or stones.

The above and further aspects and preferred embodiments of the invention are described in the following sections and in the appended claims. The subject-matter of appended claims is hereby specifically incorporated in this specification.

### DESCRIPTION OF THE DRAWINGS

FIG. 1 provides a schematic representation of the main reabsorption of phosphate in humans (left) and of the main reabsorption of oxalate in humans as found by the present inventors (right).

FIG. 2 represents serial X-ray images at (A) 30 min, (B) 90 min, and (C) 4 hours of the abdomen of a normal volunteer in standing anteroposterior direction after oral ingestion of the pharmaceutical dosage form according to an embodiment of the invention, namely two capsules illustrating the invention comprising each 250 mg of elemental lanthanum, demonstrating the release of lanthanum carbonate from the capsules in the distal part of the gastrointestinal tract.

FIG. 3 represents a graph illustrating the urinary oxalate concentration (mg/L, white bars); urinary phosphate (P) concentration (mmol/L, black bars) and the urinary calcium (Ca) concentration (mmol/L, striped bars) of a normal volunteer after oral ingestion of the pharmaceutical dosage form according to an embodiment of the invention in a dosage of 1000 mg elemental lanthanum/day, namely of capsules illustrating the invention comprising each 250 mg of elemental lanthanum (i.e., 1000 mg elemental lanthanum/day in four capsules of 250 mg elemental lanthanum: 2×250 mg in the morning, 2×250 mg in the evening).

FIG. 4 represents graphs of the results of determination of (A) urinary oxalate (mg/L), (B) urinary oxalate (mg/gr creatinine), (C) urinary calcium (mg/gr creatinine), (D) urinary phosphate (mg/gr creatinine) and (E) oxalate in serum (mg/L) before, during, and after treatment (2.5 months) with the pharmaceutical dosage form according to an embodiment of the invention in a patient with a serious form of type 1primary hyperoxaluria (Patient 1). Bold lines represent the upper limits of normal.

FIG. 5 represents graphs of the results of determination of (A) urinary oxalate (mg/L), (B) urinary oxalate (mg/gr creatinine), (C) serum oxalate (mg/L), (D) urinary calcium (mg/gr creatinine), and (E) urinary phosphate (mg/gr creatinine) before, during, and after treatment with the pharmaceutical dosage form according to an embodiment of the invention in a patient with secondary hyperoxaluria (Patient 5, idiopathic calcium oxalate urolithiasis type). Bold lines represent the upper limits of normal.

FIG. 6 provides serial X-ray images at 0 min, 60 min, 120 min, 180 min, 240 min, and 300 min of the abdomen of a normal volunteer in standing anteroposterior direction after oral ingestion of the pharmaceutical dosage form according to an embodiment of the invention, namely two capsules illustrating the invention comprising each 250 mg of elemental lanthanum, demonstrating the release of lanthanum carbonate from the capsules in the distal part of the gastrointestinal tract including ileum, cecum and colon.

FIG. 7 provides a schematic representation of the oxalate balance in a patient with a serious form of type 1 primary hyperoxaluria (Patient 1) before (left panel, Control period) and after treatment (right panel, Treatment with sdc Lanthanum Carbonate) with the pharmaceutical dosage form according to an embodiment of the invention. G.I.T: gastro-intestinal tract.

FIG. 8 represents an X-ray image of lanthanum capsules according to an embodiment of the invention.

FIG. 9 represents graphs illustrating (A) the urinary oxalate concentration (mg/g creatinine, dark grey bars) and the urinary calcium (Ca) concentration (mmol/g creatinine, light grey bars), or (B) urinary phosphate (P) concentration (mmol/L, grey bars) and serum phosphate (P) concentration (mmol/L, black bars) of a normal volunteer (male, 74 years of age, eGFR 73 ml/min/1.73 m²) after oral ingestion of Fosrenol (Shire Pharmaceuticals), i.e., uncoated lanthanum carbonate, in a dosage of 500 mg elemental lanthanum/day (2×250 mg elemental lanthanum).

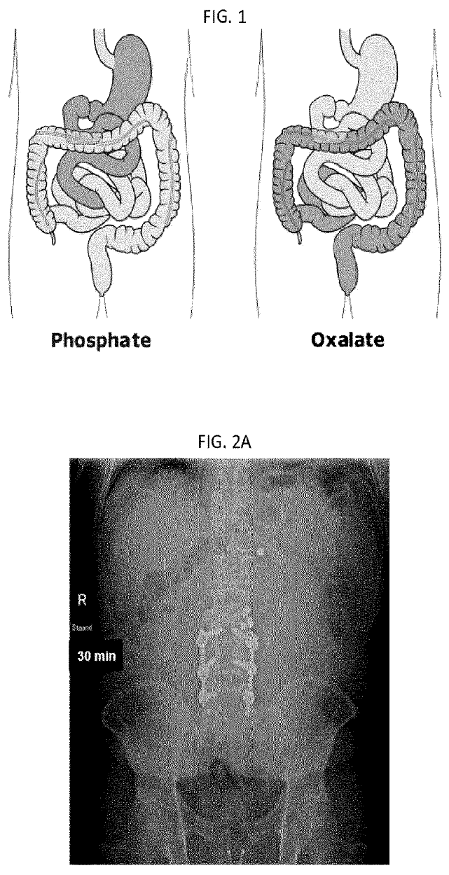

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All documents cited in the present specification are hereby incorporated by reference in their entirety. Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

By extensive experiment testing, the present inventors have found a pharmaceutical dosage form to treat one or more of hyperoxaluria, nephrolithiasis, nephrocalcinosis, or oxalosis. The pharmaceutical dosage form allows release of lanthanum in the distal part of the gastrointestinal tract, thereby avoiding interactions of lanthanum with phosphate salts in the duodenum and jejunum, whereas oxalate is chelated locally in the more distal part of the gastrointestinal tract in the form of lanthanum oxalate and calcium oxalate. These chelation products are eliminated through the stools. The overall result is a substantial decrease of oxalate absorption and decrease of the renal load, resulting in a decreased oxaluria and decreased or unchanged calciuria, thereby arresting calcium oxalate crystal formation. The phosphate balance remains unchanged in view of the intact proximal gut phosphate absorption.

Accordingly, a first aspect of the invention relates to an orally administrable pharmaceutical dosage form configured for targeted delivery to the distal part of the gastrointestinal tract of a subject, wherein the pharmaceutical dosage form comprises lanthanum or a pharmaceutically acceptable salt or oxide thereof.

Preferably, a first aspect of the invention relates to an orally administrable pharmaceutical dosage form comprising lanthanum or a pharmaceutically acceptable salt or oxide thereof, wherein the pharmaceutical dosage form is configured for targeted (or specific) delivery of the lanthanum or pharmaceutically acceptable salt or oxide thereof to one or more of the ileum, cecum, and colon of a human subject.

The pharmaceutical dosage form as taught herein is an orally administrable pharmaceutical dosage form, also referred to as oral dosage form. Advantageously, an oral dosage form is convenient, allows for improved patient adherence and relatively safe administration, and does not require sterile preparation.

The wording “oral” or “orally administrable” refers to the route of administration where a substance or composition is configured to be taken through the mouth.

The phrase “orally administrable pharmaceutical dosage form” refers to a pharmaceutical dosage form which is configured to be taken through the mouth.

The terms “pharmaceutical dosage form”, “pharmaceutical unit dose”, or “dosage form” refer to a pharmaceutical drug product in the form in which it is marketed for use, comprising a specific mixture of one or more active ingredients and one or more inactive components (e.g. excipients) in a particular configuration and apportioned into a particular dose.

The term “pharmaceutical” or “pharmaceutically acceptable” as used herein is consistent with the art and refers to being compatible with the active ingredients and not deleterious to the recipient thereof.

The terms “active ingredient” or “active component” can be used interchangeably and broadly refer to a compound or substance which, when provided in an effective amount, achieves a desired outcome. The desired outcome may be therapeutic and/or prophylactic. Typically, an active ingredient may achieve such outcome(s) through interacting with and/or modulating living cells or organisms.

The term “active” in the recitations “active ingredient” or “active component” refers to “pharmacologically active” and/or “physically active”.

In embodiments, the pharmaceutical dosage form is configured for targeted delivery to the distal part of the gastrointestinal tract of a subject.

The terms “gastrointestinal tract”, “GI tract” or “digestive tract” may be used interchangeably herein and refer to the tract or passageway of the digestive system that leads from the mouth to the anus. In humans and other animals, the GI tract contains all the major organs of the digestive system including the oesophagus, stomach, small intestine, and large intestine.

The terms “small intestine” or “small bowel” refer to the segment of the gastrointestinal tract being present between but not including the stomach and large intestine. The small intestine has three distinct regions: the duodenum, jejunum, and ileum.

The term “duodenum” refers to the first section of the small intestine. The duodenum is typically about 20-25 cm long. The duodenum receives chyme from the stomach, together with pancreatic juice containing digestive enzymes and bile from the gall bladder.

The term “jejunum” refers to the midsection of the small intestine, connecting the duodenum to the ileum. The jejunum is typically about 2.5 m long. The jejunum contains the circular folds also known as plicae circulares and villi that increase its surface area.

The terms “proximal small intestine” or “early small intestine” refer to the first section and midsection of the small intestine including the duodenum and jejunum.

The term “ileum” refers to the last section of the small intestine. The ileum is about 3 m long. The ileum contains villi similar to the jejunum.

The terms “distal small intestine” or “late small intestine” refer to the last section of the small intestine including the ileum. The terms “ileum”, “distal small intestine” or “late small intestine” may be used interchangeably herein.

The terms “large intestine” or “large bowel” may be used interchangeably and refer to the last segment of the gastrointestinal tract. The large intestine is subdivided into the cecum, colon, rectum, and anal canal.

The “cecum” refers to a pouch within the peritoneum that connects the ileum and the colon, in particular the ascending colon. The cecum is separated from the ileum by the ileocecal valve (ICV) or Bauhin's valve. The cecum is separated from the colon by the cecocolic junction.

The term “colon” refers to the longest segment of the large intestine. The colon comprises four sections, i.e., the ascending colon, transverse colon, descending colon, and sigmoid colon, which sections turn at the colic flexures.

The terms “early colon” or “proximal colon” as used herein refer to the ascending colon.

In embodiments of the products (e.g. pharmaceutical dosage form), uses, or methods as taught herein, the distal part of the gastrointestinal tract may be the ileum, the cecum, the colon, or a combination thereof. In embodiments, the distal part of the gastrointestinal tract may be the ileum, the colon, or a combination thereof. In embodiments, the distal part of the gastrointestinal tract may be the ileum and the colon. In embodiments, the distal part of the gastrointestinal tract may be the ileum, the cecum, the early colon, or a combination thereof. In embodiments, the distal part of the gastrointestinal tract may be the ileum, the early colon, or a combination thereof. In embodiments, the distal part of the gastrointestinal tract may be the ileum and the early colon.

In embodiments, the pharmaceutical dosage form may be configured for targeted delivery to the ileum, cecum, and colon of a subject, in particular of a human subject. In embodiments of the products, uses, or methods as taught herein, the pharmaceutical dosage form may be configured for targeted delivery of the lanthanum or pharmaceutically acceptable salt or oxide thereof to one or more of the ileum, cecum, and colon of a subject, preferably of a human subject. In embodiments of the products, uses, or methods as taught herein, the pharmaceutical dosage form may be configured for targeted delivery of the lanthanum or pharmaceutically acceptable salt or oxide thereof to one or more of the ileum, cecum, and ascending colon of a subject, preferably of a human subject. Specific delivery of the lanthanum or pharmaceutically acceptable salt or oxide thereof to one or more of the ileum, cecum, and colon of a subject advantageously allows treatment of primary or secondary hyperoxaluria while avoiding interactions of lanthanum with phosphate salts in the duodenum and jejunum and avoiding unwanted phosphate depletion.

In embodiments, the pharmaceutical dosage form may be configured for targeted delivery to the ileum and early colon of a subject.

In embodiments, targeted delivery of the lanthanum or pharmaceutically acceptable salt or oxide thereof to one or more of the ileum, cecum, or colon may be determined by imaging such as X-ray imaging or computed tomography (CT) including ultra-low dose CT (ULDCT). As illustrated in the example section (Example 2), the targeted delivery of lanthanum can easily be verified by imaging (e.g. non-invasive X-ray imaging). For instance, serial images may be taken of the abdomen of a subject (e.g. a healthy volunteer or a diseased subject) in standing anteroposterior direction after oral ingestion of one or more pharmaceutical dosage forms according to an embodiment of the invention (e.g. capsules comprising 250 mg elemental lanthanum each). Images may be taken every 5 min, 10 min, 15 min, 20 min, 25 min, 30 min or every hour for at least 360 min (6 hours). The pharmaceutical dosage form as taught herein can be seen as radiopaque structures on X-ray imaging or CT imaging. As shown in the example section, the localisation of the release of lanthanum carbonate from the pharmaceutical dosage form as taught herein is in the ileum, cecum and colon.

The phrases “targeted delivery of the lanthanum or pharmaceutically acceptable salt or oxide thereof to one or more of the ileum, cecum, or colon” or “specific delivery of the lanthanum or pharmaceutically acceptable salt or oxide thereof to one or more of the ileum, cecum, or colon” refer to the (substantially) exclusive delivery (or release) of the lanthanum or pharmaceutically acceptable salt or oxide thereof to one or more of the ileum, cecum, or colon. Accordingly, the targeted or specific delivery as taught herein means that there is (substantially) no delivery (or release) of the lanthanum or pharmaceutically acceptable salt or oxide thereof outside the ileum, cecum, and colon. The targeted or specific delivery as taught herein means that there is (substantially) no delivery (or release) of the lanthanum or pharmaceutically acceptable salt or oxide thereof to the proximal parts of the gastrointestinal tract, or in other words that there is (substantially) no delivery (or release) of the lanthanum or pharmaceutically acceptable salt or oxide thereof to the stomach, duodenum, or jejunum.

Delivery systems configured for targeted delivery to the distal part of the GI tract are known in the art and use the difference in the pH along the GI tract, the presence of colonic microflora, enzymes, or a combination of one or more thereof to achieve targeting to the distal part of the GI tract.

Delivery systems configured for targeted delivery of the pharmaceutical dosage form as taught herein to the distal part of the GI tract may include coatings, colon-specific biodegradable delivery systems, matrix-based systems, time-release systems, multiparticulate systems, polysaccharide-based delivery systems, pressure controlled delivery systems, osmotic controlled delivery systems, Pulsincap systems, and combinations thereof. These systems are known in the art for instance as described in Amidon et al. (2015, AAPS PharmSciTech, 16, 4) and Philip et al. (2010, OMJ. 25, 70-78).

Briefly, colon targeting by coating is typically based on incorporating the drug such as herein the pharmaceutical dosage form comprising the lanthanum or pharmaceutically acceptable salt or oxide thereof in a pH-sensitive polymer to allow for delayed release by protecting the drug from the acidic pH of the stomach and proximal small intestine. The polymers then break down in the more basic pH of the ileum, thus providing a targeted drug delivery to the ileum and the colon. Some examples of commonly used pH-sensitive polymers in the design of ileum and colon-targeted drug delivery systems include methacrylic acid based polymers, also known as Eudragit® polymers (Evonik Industries, Belgium).

Colon-specific biodegradable delivery systems are based on coating the drug such as herein the pharmaceutical dosage form comprising the lanthanum or pharmaceutically acceptable salt or oxide thereof by polymers to protect it from degradation by enzymes in the stomach and proximal small intestine while permitting degradation by enzymes and release of the drug in the distal part of the GI tract.

Matrix-based systems are based on embedding the drug such as herein the pharmaceutical dosage form comprising the lanthanum or pharmaceutically acceptable salt or oxide thereof in polymer matrices to trap it and release it in the distal part of the GI tract. The matrices may be pH-sensitive or biodegradable matrices.

Time-release systems are based on the drug such as herein the pharmaceutical dosage form comprising the lanthanum or pharmaceutically acceptable salt or oxide thereof being released in the distal part of the GI tract after a specified amount of time. This approach is dependent on the transit time through the small intestine, which is and known to vary.

Multiparticulate systems such as microspheres can be loaded with the active ingredient such as the lanthanum or pharmaceutically acceptable salt or oxide thereof. For example, microspheres can be coated with pH-sensitive polymers such as Eudragit® polymers. The multiparticulate systems have a smaller particle size compared to single-unit systems, and they allow to reach the distal part of the GI tract more quickly since they pass through the GI tract more easily.

Polysaccharide-based delivery systems are based on the use of one or more polysaccharides such as pectin, chitosan, chondroitin sulfate, galactomannan, or amylose as a thin film coating, compression coating, or matrix for the delivery of the active ingredient to the distal part of the GI tract.

Pressure controlled delivery systems are based on colonic luminal pressure. Peristaltic motion causes the luminal pressure of the large intestine to increase more than that of the small intestine because its contents are more viscous due to the reabsorption of water.

Osmotic controlled delivery systems such as the OROS-CT are systems regulated by osmotic pressure.

Pulsincap systems are based on the integration of a timed-release system with pH-sensitive properties.

In embodiments of the products, uses, or methods as taught herein, the pharmaceutical dosage form may comprise a coating configured for targeted delivery to the distal part of the gastrointestinal tract of a subject. In embodiments of the products, uses, or methods as taught herein, the pharmaceutical dosage form may comprise a coating configured for targeted delivery of the lanthanum or pharmaceutically acceptable salt or oxide thereof to one or more of the ileum, cecum, or colon of a subject, in particular of a human subject.

The term “coating” refers to a covering that is applied to the surface of an object. Particularly herein, the coating may be applied to the surface of the pharmaceutical dosage form.

In embodiments, the coating may be a thin film coating or a compression coating.

The term “thin film coating” refers to a thin polymer-based coat that is typically sprayed on to a solid pharmaceutical dosage form, such as a tablet, capsule, pellet, or granule. The film coating can impact the appearance (e.g., colour, gloss, and branding), i.e., non-functional film coating, and/or the pharmacokinetics (i.e., functional film coating) of the pharmaceutical dosage form. A thin film coating may be obtained by a dip coating process such as the process as taught herein.

The terms “compression coating”, “dry coating”, “press coating” “solvent-less coating” or “tablet-in-a-tablet” refer to a coating technique where the core tablet (e.g., containing the lanthanum or pharmaceutically acceptable salt or oxide thereof) is coated with a coating material (e.g., powder) on a compression machine. This technique advantageously avoids the use of solvents, requires a relatively short manufacturing process and allows greater weight gain to the core tablet. The compression coating can impact the pharmacokinetics (i.e., functional coating) of the pharmaceutical dosage form.

In embodiments, the coating is a functional coating, thereby allowing to target the pharmaceutical dosage form for delivery to the distal part of the GI tract. In embodiments, the coating is a functional coating, thereby allowing to target the lanthanum or pharmaceutically acceptable salt or oxide thereof to one or more of the ileum, cecum, or colon.

In embodiments, the coating may comprise (including consist essentially of or consist of) one or more materials configured for targeted delivery to the distal part of the GI tract. In embodiments, the coating may comprise (including consist essentially of or consist of) one or more materials configured for targeted delivery of the lanthanum or pharmaceutically acceptable salt or oxide thereof to one or more of the ileum, cecum, or colon.

In embodiments of the products, uses, or methods as taught herein, instead of comprising a coating configured for targeted delivery to the distal part of the gastrointestinal tract of a subject, the pharmaceutical dosage form may comprise one or more materials configured for targeted delivery to the distal part of the GI tract. In embodiments of the products, uses, or methods as taught herein, instead of comprising a coating configured for targeted delivery to one or more of the ileum, cecum, or colon of a subject, the pharmaceutical dosage form may comprise one or more materials configured for targeted delivery to one or more of the ileum, cecum, or colon of a subject.

In embodiments, the coating or the dosage form may comprise a pH sensitive polymer, a biodegradable polymer, a polysaccharide, or a combination thereof. In preferred embodiments, the coating or the dosage form may comprise a pH sensitive polymer. In embodiments, the coating or the dosage form may comprise a polymer which starts disintegrating (i.e., breaks down) at pH conditions in the distal part of the GI tract, e.g. in the ileum and the colon, such as at a pH of at least 7.0. In embodiments, the coating or the dosage form may comprise a polymer which does not disintegrate at pH conditions in the stomach and the early small intestine (i.e., jejunum and duodenum), such as at a pH of below 7.0, at a pH of below 6.8, or at a pH of below 6.5.

In embodiments, the coating or the dosage form is substantially insoluble at a pH of less than a range of between about 7.0 to about 8.0. In embodiments, the coating or the dosage form is substantially soluble in the pH range of about 7.0 to about 8.0. In embodiments, the coating or the dosage form is substantially soluble in the pH range of about 6.5 to about 8.0. In embodiments, the coating or the dosage form is substantially soluble in the pH range of about 6.0 to about 8.0.

In embodiments, the coating or dosage form may comprise (including consist essentially of or consist of) a polymer configured for targeted delivery to the distal part of the GI tract. In embodiments, the coating or dosage form may comprise (including consist essentially of or consist of) a polymer configured for targeted delivery of the lanthanum or pharmaceutically acceptable salt or oxide thereof to one or more of the ileum, cecum, or colon.

In embodiments, polymers configured for targeted delivery to the distal part of the GI tract, e.g. to the ileum, cecum, and/or colon, of a subject include one or more polymers selected from the group consisting of copolymers of methacrylic acid and methyl methacrylate, copolymers of methacrylic acid, methyl acrylate and methyl methacrylate, copolymers of methacrylic acid and ethyl acrylate, and copolymers of methacrylic acid and ethyl acrylate to which a monomer of methyl acrylate has been added during polymerization. In embodiments, polymers configured for targeted delivery to the distal part of the GI tract, e.g. to the ileum, cecum, and/or colon, of a subject include one or more polymers selected from the group consisting of copolymers of methacrylic acid and methyl methacrylate, and copolymers of methacrylic acid, methyl acrylate and methyl methacrylate.

Suitable examples of polymers configured for targeted delivery to the distal part of the GI tract, e.g. to the ileum, cecum, and/or colon, include aqueous based methacrylic acid copolymers such as the Eudragit® polymers (Evonik Industries, Belgium). For instance, suitable polymers for targeted delivery to the distal part of the GI tract, such as the ileum and the colon, include Eudragit® S100, Eudragit® S12,5, Eudragit® FS 30D, Eudragit® FS 100, Eudragit® L100, Eudragit® L12,5, alone or in combinations. These polymers advantageously solubilize above pH 7.0 (e.g., Eudragit® S100, S12,5, FS 30D, FS 100), or above pH 6.0 (e.g., Eudragit® L100, Eudragit® L12,5).

In embodiments of the products, uses, or methods as taught herein, the coating or the dosage form may comprise (including consist essentially of or consist of) a copolymer of methacrylic acid and C₁₋₄alkylester of methacrylic acid. In embodiments, the coating or the dosage form may comprise of a copolymer of methacrylic acid and methyl ester of methacrylic acid (i.e., methyl methacrylate). In embodiments, the coating or the dosage form may comprise a copolymer of methacrylic acid and ethyl ester of methacrylic acid (i.e., ethyl methacrylate). In embodiments, the coating or the dosage form may comprise a copolymer of methacrylic acid and propyl ester of methacrylic acid (e.g., n-propyl methacrylate). In embodiments, the coating or the dosage form may comprise a copolymer of methacrylic acid and butyl ester of methacrylic acid (e.g., n-butyl methacrylate).

In embodiments of the products, uses, or methods as taught herein, the coating or the dosage form may comprise a copolymer of methacrylic acid and methyl methacrylate in a ratio of 2:1 to 1:2, such as in a ratio of 1:1. In preferred embodiments, the coating or the dosage form may comprise, consist essentially of, or consist of a copolymer of methacrylic acid and methyl methacrylate in a ratio of 1:2. Such a copolymer is for instance Eudragit® S100.

In embodiments, the coating may further comprise one or more additives or auxiliary agents such as a solvent, a plasticizer, a carrier, a surfactant, a thickener, a buffering agent, an antioxidant, a preservative, an aroma, or a colorant. Suitable auxiliary agents are known in the art.

In embodiments of the products, uses, or methods as taught herein, the pharmaceutical dosage form may comprise at least three layers of the coating. In embodiments, the pharmaceutical dosage form may comprise at least four layers, at least five layers, or at least six layers of the coating. Preferably, the pharmaceutical dosage form comprises at least four layers such as four layers of the coating. The at least four layers such as the four layers of the coating advantageously allows to protect the pharmaceutical dosage form in the proximal part of the GI tract and allows to deliver the pharmaceutical dosage form and release the lanthanum or pharmaceutically acceptable salt or oxide thereof in the distal part of the GI tract, such as in the ileum, cecum and/or colon.

In embodiments of the products, uses, or methods as taught herein, the thickness of the (total) coating may be at least 20.0 μm, such as at least 21.0 μm, at least 22.0 μm, at least 23.0 μm, at least 24.0 μm, at least 25.0 μm, at least 26.0 μm, at least 27.0 μm, at least 28.0 μm, at least 29.0 μm, at least 30.0 μm, at least 31.0 μm, at least 32.0 μm, at least 33.0 μm, at least 34.0 μm, or at least 35.0 μm. In embodiments, the thickness of the coating may be from about 20.0 μm to about 100.0 μm. For instance, the thickness of the coating may be from about 25.0 μm to about 60.0 μm, from about 30.0 μm to about 50.0 μm, or from about 35.0 μm to about 40.0 μm. As shown in the example section, such thicknesses advantageously allow delivery of the pharmaceutical dosage form and release of lanthanum carbonate in the distal part of the gastrointestinal tract, such as in the ileum, cecum and/or colon. The thickness of the (total) coating may be measured using microscopy or a micrometer (also known as a micrometer screw gauge or manual screw micrometer). The thickness of the (total) coating may be calculated by determining the thickness of the coated dosage form and the thickness of the uncoated dosage form and subtracting the thickness of the uncoated capsule from the thickness of the coated capsule.

In embodiments of the products, uses, or methods as taught herein, the thickness of each layer of the coating may be at least 4.0 μm, such as at least 5.0 μm, at least 6.0 μm, or at least 7.0 μm. In embodiments, the thickness of each layer of the coating may be from about 4.0 μm to about 25.0 μm. For instance, the thickness of each layer of the coating may be from about 4.0 μm to about 20.0 μm, from about 5.0 μm to about 15.0 μm, or from about 6.0 μm to about 10.0 μm.

In embodiments of the products, uses, or methods as taught herein, the pharmaceutical dosage form may comprise at least three layers such as four layers of coating, and the thickness of each layer may be at least 4.0 μm, such as at least 5.0 μm, at least 6.0 μm, or at least 7.0 μm. In embodiments, the pharmaceutical dosage form may comprise at least three layers such as four layers of coating, and the thickness of each layer may be from about 4.0 μm to about 25.0 μm. For instance, the pharmaceutical dosage form may comprise at least three layers such as four layers of coating, and the thickness of each layer of the coating may be from about 4.0 μm to about 20.0 μm, from about 5.0 μm to about 15.0 μm, or from about 6.0 μm to about 10.0 μm. Such coating advantageously allows to protect the pharmaceutical dosage form in the proximal part of the GI tract and allows to deliver the pharmaceutical dosage form and release the lanthanum or pharmaceutically acceptable salt or oxide thereof in the distal part of the GI tract, such as in the ileum, cecum and/or colon.

In embodiments of the products, uses, or methods as taught herein, the pharmaceutical dosage form may be a solid, semisolid, or liquid dosage form. For example, for oral administration, the pharmaceutical dosage form includes pills, tablets, granules, capsules, aqueous, alcoholic or oily solutions, syrups, emulsions, or suspensions. For example, the pharmaceutical dosage form may also be a colloidal dispersion system. Colloidal dispersion systems include, but are not limited to, macromolecule complexes, nanocapsules, microspheres, beads and lipid-based systems including oil-in-water emulsions, micelles, mixed micelles, liposomes and lipid: compound complexes of uncharacterized structure. A preferred colloidal dispersion system is a plurality of liposomes. Liposomes are microscopic spheres having an aqueous core surrounded by one or more outer layers made up of lipids arranged in a bilayer configuration (see, Chonn et al., 1995, Current Op. Biotech. 6, 698-708). Likewise, microparticulate or nanoparticulate polymeric bead dosage forms may be used in the pharmaceutical dosage form provided herein.

In embodiments of the products, uses, or methods as taught herein, the pharmaceutical dosage form may be a liquid dosage form such as an aqueous, alcoholic or oily solution, a syrup, an emulsion, or a suspension. For instance, the pharmaceutical dosage form may be microspheres suspended in a solution.

In embodiments of the products, uses, or methods as taught herein, the pharmaceutical dosage form may be a solid or semisolid dosage form. In embodiments, the pharmaceutical dosage form may be a pill, a pellet, a tablet, a granule, or a capsule such as a hard or soft capsule.

In embodiments of the products, uses, or methods as taught herein, the pharmaceutical dosage form may be a capsule or a tablet. Preferably, the pharmaceutical dosage form may be a capsule, such as a capsule filled with a composition comprising the lanthanum or pharmaceutically acceptable salt or oxide thereof; preferably wherein the lanthanum or the pharmaceutically acceptable salt or oxide thereof is in powder form.

In embodiments of the products, uses, or methods as taught herein, the capsule may be made of one or more materials selected from the group consisting of cellulose acetate phthalate (CAP), hydroxypropyl methylcellulose (HPMC), gelatin, pullulan, starch, a water-soluble polyvinyl derivative, a polyethylene glycol, and a copolymer of C₁₋₄alkylester of (meth)acrylic acid and (meth)acrylic acid.

In embodiments, the capsule may have size 000, size 00E, size 00, size 0E, size 0, size 1, size 2, size 3, size 4, or size 5. In embodiments, the capsule may have size 00, size 0E, size 0, size 1, size 2, size 3, size 4, or size 5.

In embodiments, the capsule may have size 00 and may comprise 500 mg to 600 mg of elemental lanthanum, for instance the capsule may have size 00 and may comprise 560 mg of elemental lanthanum. In embodiments, the capsule may have size 0 and may comprise 400 mg to 500 mg of elemental lanthanum, for instance the capsule may have size 0 and may comprise 420 mg of elemental lanthanum. In embodiments, the capsule may have size 1 and may comprise 250 mg to 400 mg of elemental lanthanum, for instance the capsule may have size 1 and may comprise 295 mg of elemental lanthanum. In embodiments, the capsule may have size 2 and may comprise 200 mg to 300 mg of elemental lanthanum, for instance the capsule may have size 2 and may comprise 230 mg of elemental lanthanum.

In embodiments of the products, uses, or methods as taught herein, the capsule may comprise a coating configured for targeted delivery to the distal part of the gastrointestinal tract of a subject; preferably wherein the coating comprises a pH sensitive polymer, a biodegradable polymer, a polysaccharide, or a combination thereof. In embodiments of the products, uses, or methods as taught herein, the capsule may comprise a coating configured for targeted delivery of the lanthanum or pharmaceutically acceptable salt or oxide thereof to one or more of the ileum, cecum, or colon of a subject; preferably wherein the coating comprises a pH sensitive polymer, a biodegradable polymer, a polysaccharide, or a combination thereof. As shown in the experimental section, a coated capsule as taught herein allows to traverse the proximal gastrointestinal tract and reach the target site namely the ileum and colon for release of the lanthanum or pharmaceutically acceptable salt or oxide thereof. The GI tract physiology is complex and has a wide range of pH values, fluid volumes, and transit times, while the presence of food and metabolic enzymes also increases the physiological complexity. Despite these factors, a coated capsule as taught herein allows the reliable and efficient delivery of the lanthanum or pharmaceutically acceptable salt or oxide thereof to the distal part of the GI tract including the ileum and colon.

In embodiments, the capsule may comprise one or more materials configured for targeted delivery to the distal part of the gastrointestinal tract of a subject; preferably wherein the capsule comprises a pH sensitive polymer, a biodegradable polymer, a polysaccharide, or a combination thereof. In embodiments, the capsule may comprise one or more materials configured for targeted delivery of the lanthanum or pharmaceutically acceptable salt or oxide thereof to one or more of the ileum, cecum, or colon of a subject; preferably wherein the capsule comprises a pH sensitive polymer, a biodegradable polymer, a polysaccharide, or a combination thereof.

In embodiments of the products, uses, or methods as taught herein, the capsule may comprise at least three layers of the coating. In embodiments, the capsule may comprise at least four layers, at least five layers, or at least six layers of the coating. Preferably, the capsule comprises at least four layers such as four layers of the coating. The at least four layers of the coating such as the four layers of the coating advantageously allow to protect the capsule in the proximal part of the GI tract against degradation and allows to deliver the capsule in the distal part of the GI tract, e.g. in the ileum, cecum, and/or colon, and release the lanthanum or pharmaceutically acceptable salt or oxide thereof.

The pharmaceutical dosage form as taught herein comprises lanthanum or a pharmaceutically acceptable salt or oxide thereof. In embodiments of the products, uses, or methods as taught herein, the pharmaceutically acceptable salt or oxide of lanthanum may be lanthanum carbonate or lanthanum oxide; preferably wherein the pharmaceutical dosage form comprises lanthanum carbonate.

The term “lanthanum” refers to a chemical element with the symbol La and atomic number 57. Lanthanum has a molar mass of 138.91 g/mol.

The phrase “pharmaceutically acceptable salt thereof” refers to a salt of lanthanum derived from the combination of lanthanum and an organic or inorganic acid (acid addition salts) or an organic or inorganic base (base addition salts).

In embodiments of the products, uses, or methods as taught herein, the pharmaceutically acceptable salt of lanthanum may be lanthanum carbonate.

The term “lanthanum carbonate” refers to a compound with formula La₂(CO₃)₃. It is a salt formed by lanthanum(III) cations and carbonate anions. Lanthanum carbonate has CAS number 587-26-8. Lanthanum carbonate has a molar mass of 457.838 g/mol. It can be calculated that 1000 mg (1 g) of lanthanum carbonate corresponds to about 606.8 mg elemental lanthanum, or that 1000 mg (1 g) of elemental lanthanum corresponds to 1648 mg lanthanum carbonate.

Lanthanum carbonate is commercially available as a phosphate binder and is sold under the trade name Fosrenol (Shire Pharmaceuticals, Dublin, Ireland).

In embodiments of the products, uses, or methods as taught herein, the pharmaceutically acceptable oxide of lanthanum may be lanthanum oxide.

The terms “lanthanum oxide” or “lanthana” refer to a compound with formula La₂O₃. It is an inorganic compound containing lanthanum (III) and oxygen. Lanthanum oxide has CAS number 1312-81-8.

In embodiments, the lanthanum or pharmaceutically acceptable salt or oxide thereof may be lanthanum, lanthanum carbonate, or lanthanum oxide.

When reference is made to lanthanum, it is meant to refer to elemental lanthanum. However, when a pharmaceutical dosage form is said to comprise a specific amount of elemental lanthanum, it is meant that the pharmaceutical dosage form comprises the specified amount of elemental lanthanum, either as elemental lanthanum or as a pharmaceutically acceptable salt or oxide thereof. For instance, when a pharmaceutical dosage form is said to comprise 250 mg of elemental lanthanum, it is meant that the pharmaceutical dosage form comprises 250 mg of elemental lanthanum, either as elemental lanthanum or in a pharmaceutically acceptable salt or oxide thereof. For instance, a pharmaceutical dosage form said to comprise 250 mg of elemental lanthanum may comprise 250 mg of elemental lanthanum or about 412 mg of lanthanum carbonate.

In embodiments of the products, uses, or methods as taught herein, the pharmaceutical dosage form may comprise 100 mg to 4000 mg (4 g) of elemental lanthanum. In embodiments, the pharmaceutical dosage form may comprise 100 mg to 3500 mg (3.5 g), 100 mg to 3000 mg (3 g), 100 mg to 2500 mg (2.5 g), 100 mg to 2000 mg (2 g), 100 mg to 1500 mg (1.5 g), or 100 mg to 1000 mg (1 g) of elemental lanthanum. In embodiments of the products, uses, or methods as taught herein, the pharmaceutical dosage form may comprise 100 mg to 750 mg, 100 mg to 500 mg, or 100 mg to 250 mg of elemental lanthanum. In embodiments of the products, uses, or methods as taught herein, the pharmaceutical dosage form, such as the capsule, may comprise 200 mg to 750 mg of elemental lanthanum, such as 250 mg to 600 mg elemental lanthanum. In embodiments, the pharmaceutical dosage form, such as the capsule, may comprise 500 mg to 600 mg elemental lanthanum. In embodiments, the pharmaceutical dosage form, such as the capsule, may comprise 400 mg to 500 mg elemental lanthanum. In embodiments, the pharmaceutical dosage form, such as the capsule, may comprise 250 mg to 400 mg elemental lanthanum. In embodiments, the pharmaceutical dosage form, such as the capsule, may comprise 200 mg to 300 mg elemental lanthanum. In embodiments, the pharmaceutical dosage form may comprise at least 210 mg, at least 220 mg, at least 230 mg, at least 240 mg, at least 250 mg, at least 260 mg, at least 270 mg, at least 280 mg, at least 290 mg, at least 300 mg, at least 310 mg, at least 320 mg, at least 330 mg, at least 340 mg, at least 350 mg, at least 360 mg, at least 370 mg, at least 380 mg, at least 390 mg, at least 400 mg, at least 410 mg, at least 420 mg, at least 430 mg, at least 440 mg, at least 450 mg, at least 460 mg, at least 470 mg, at least 480 mg, at least 490 mg, at least 500 mg, at least 510 mg, at least 520 mg, at least 530 mg, at least 540 mg, at least 550 mg, at least 560 mg, at least 570 mg, at least 580 mg, at least 590 mg, at least 600 mg, at least 610 mg, at least 620 mg, at least 630 mg, at least 640 mg, at least 650 mg, at least 660 mg, at least 670 mg, at least 680 mg, at least 690 mg, at least 700 mg, at least 710 mg, at least 720 mg, at least 730 mg, at least 740 mg, or at least 750 mg of elemental lanthanum.

In embodiments, the pharmaceutical dosage form as taught herein may comprise, such as may be filled with, lanthanum carbonate, preferably in powder form. In embodiments, the capsule as taught herein may comprise, such as may be filled with, lanthanum carbonate, preferably in powder form. In embodiments, the pharmaceutical dosage form as taught herein may comprise, such as may be filled with, about 200 mg to about 2 g of lanthanum carbonate, preferably in powder form. In embodiments, the capsule as taught herein may comprise, such as may be filled with, about 200 mg to about 2 g lanthanum carbonate, preferably in powder form.

In embodiments, the pharmaceutical dosage form as taught herein may comprise, such as may be filled with, lanthanum or a pharmaceutically acceptable salt or oxide thereof for release in the distal part of the GI tract. In embodiments, the pharmaceutical dosage form as taught herein may comprise, such as may be filled with, lanthanum or a pharmaceutically acceptable salt or oxide thereof for release in one or more of the ileum, cecum, or colon. Advantageously, the lanthanum or pharmaceutically acceptable salt or oxide thereof within the dosage form is delivered to the distal part of the GI tract without leakage of the lanthanum or pharmaceutically acceptable salt or oxide thereof in the proximal part of the gastrointestinal tract positioned before the ileum. In embodiments, the pharmaceutical dosage form as taught herein may provide for at least 80% of the lanthanum or pharmaceutically acceptable salt or oxide thereof to reach the distal part of the GI tract, preferably at least 85%, at least 90%, at least 95%, or at least 97%, or more such as at least 98%, at least 99%, or 100% of the lanthanum or pharmaceutically acceptable salt or oxide thereof to reach the distal part of the GI tract, e.g., the ileum, cecum, and/or colon.

In embodiments, the pharmaceutical dosage form may be a capsule filled with a composition comprising the lanthanum or pharmaceutically acceptable salt or oxide thereof.

In embodiments, the composition comprising the lanthanum or pharmaceutically acceptable salt or oxide thereof may comprise one or more excipients.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, antioxidants, tonicity controlling agents, absorption delaying agents, and the like. The use of such media and agents for pharmaceutical active ingredients is well known in the art. Except as far as any conventional media or agent is incompatible with the active ingredient, its use in the pharmaceutical dosage form may be contemplated.

In embodiments, the composition comprising the lanthanum or pharmaceutically acceptable salt of oxide thereof may comprise one or more excipients including dextranes (hydrated), colloidal water-free silica and magnesium stearate.

In embodiments, the pharmaceutical dosage form may be a capsule filled with a composition comprising the lanthanum or pharmaceutically acceptable salt or oxide thereof, wherein the lanthanum or the pharmaceutically acceptable salt or oxide thereof or the composition is in powder form.

The term “powder” refers to a dry, bulk solid composed of many very fine particles that may flow freely when shaken or tilted.

In embodiments of the products, uses, or methods as taught herein, the pharmaceutical dosage form may be a capsule filled with a composition comprising the lanthanum or pharmaceutically acceptable salt or oxide thereof, and coated with a copolymer of methacrylic acid and C₁₋₄alkylester of methacrylic acid, preferably wherein the composition comprising the lanthanum or pharmaceutically acceptable salt or oxide thereof is in powder form. Preferably, the capsule may comprise 100 mg to 1 g of lanthanum such as 250 mg to 500 mg of lanthanum.

In embodiments, the pharmaceutical dosage form may be a capsule filled with a composition comprising the lanthanum or pharmaceutically acceptable salt or oxide thereof, such as lanthanum carbonate, and coated with at least four layers of a copolymer of methacrylic acid and C₁₋₄alkylester of methacrylic acid, preferably wherein the composition comprising the lanthanum or pharmaceutically acceptable salt or oxide thereof is in powder form. In embodiments, the pharmaceutical dosage form may be a capsule filled with a composition comprising the lanthanum or pharmaceutically acceptable salt or oxide thereof, such as lanthanum carbonate, and coated with at least four layers of a copolymer of methacrylic acid and methyl methacrylate, preferably wherein the composition comprising the lanthanum or pharmaceutically acceptable salt or oxide thereof is in powder form. In embodiments, the pharmaceutical dosage form may be a capsule filled with a composition comprising the lanthanum or pharmaceutically acceptable salt or oxide thereof, such as lanthanum carbonate, and coated with at least four layers of a copolymer of methacrylic acid and methyl methacrylate in a ratio of 1:2, preferably wherein the composition comprising the lanthanum or pharmaceutically acceptable salt or oxide thereof is in powder form. Preferably, the capsule may comprise 100 mg to 1 g of lanthanum such as 250 mg to 500 mg of lanthanum. As shown in the example section, such a pharmaceutical dosage form when used as a medicament advantageously decreased urinary oxalate levels, while maintaining or even lowering urinary calcium levels, and without changing urinary phosphate levels. The present pharmaceutical dosage form thereby allows to arrest calcium oxalate crystal formation in the tubular fluid of the kidney and urine without causing phosphate depletion. Since calcium oxalate crystals are the first critical step in the formation of renal calculi, eventually causing nephrolithiasis, the present pharmaceutical dosage form advantageously allows to reduce hyperoxaluria and even avoid nephrolithiasis, nephrocalcinosis, and oxalosis, in particular nephrolithiasis, nephrocalcinosis, and oxalosis caused by calcium oxalate.

A second aspect provides the pharmaceutical dosage form as defined herein, for use as a medicament in a subject, in particular a human subject. Accordingly, an aspect provides an orally administrable pharmaceutical dosage form configured for targeted delivery to the distal part of the gastrointestinal tract of a subject, wherein the pharmaceutical dosage form comprises lanthanum or a pharmaceutically acceptable salt or oxide thereof, for use as a medicament in the subject. Preferably, an aspect provides an orally administrable pharmaceutical dosage form comprising lanthanum or a pharmaceutically acceptable salt or oxide thereof, wherein the pharmaceutical dosage form is configured for targeted delivery of the lanthanum or pharmaceutically acceptable salt or oxide thereof to one or more of the ileum, cecum, or colon of a subject, for use as a medicament in the subject. Preferably, the subject is a human subject.

A further aspect provides the pharmaceutical dosage form as defined herein, for use in the treatment of hyperoxaluria in a subject. Hence, a further aspect provides an orally administrable pharmaceutical dosage form configured for targeted delivery to the distal part of the gastrointestinal tract of a subject, wherein the pharmaceutical dosage form comprises lanthanum or a pharmaceutically acceptable salt or oxide thereof, for use in the treatment of hyperoxaluria in the subject. Preferably, a further aspect provides an orally administrable pharmaceutical dosage form comprising lanthanum or a pharmaceutically acceptable salt or oxide thereof, wherein the pharmaceutical dosage form is configured for targeted delivery of the lanthanum or pharmaceutically acceptable salt or oxide thereof to one or more of the ileum, cecum, or colon of a subject, for use in the treatment of hyperoxaluria in the subject.

In embodiments, the hyperoxaluria may be primary hyperoxaluria or secondary hyperoxaluria. Preferably, the subject is a human subject.

Related aspects provide:
a method of treating hyperoxaluria, such as primary or secondary hyperoxaluria, in a subject in need of such a treatment, comprising administering, preferably orally administering, a therapeutically effective amount of a pharmaceutical dosage form as taught herein to the subject.
a method of treating hyperoxaluria, such as primary or secondary hyperoxaluria, in a human subject in need of such a treatment, comprising administering, preferably orally administering, a therapeutically effective amount of a pharmaceutical dosage form as taught herein to the subject.
the use of a pharmaceutical dosage form as taught herein for the manufacture of a medicament for the treatment of hyperoxaluria, such as primary or secondary hyperoxaluria, in a subject.
the use of a pharmaceutical dosage form as taught herein for the manufacture of a medicament for the treatment of hyperoxaluria, such as primary or secondary hyperoxaluria, in a human subject.

The terms “hyperoxaluria” or “Bird's disease” refer to a disease or condition characterised by an excessive urinary excretion of oxalate.

The term “oxalate” refers to an anion with the formula C₂O₄²⁻. The IUPAC name is ethanedioate.

The term “primary hyperoxaluria (PH)” refers to a rare, genetic (inherited) disorder of the liver in which the liver either does not produce enough enzymes to prevent the overproduction of oxalate or the enzymes do not work properly. There are three subtypes of primary hyperoxaluria, each of which involves a genetic defect of a different enzyme in the liver and all of which play a role in the overproduction of oxalate.

Primary hyperoxaluria type 1 (PH1) is the most common and severe form of PH. PH1 accounts for approximately 80% of the cases of PH. PH1 is caused by a defect in the vitamin B6 dependent hepatic peroxisomal enzyme, Alanine Glyoxalate Aminotransferase (AGT) which catalyses the transamination of L-alanine and glyoxalate to pyruvate and glycine. The enzyme defect has been attributed to a mutation in the AGXT gene located on chromosome 2.

Primary hyperoxaluria type 2 (PH2) represents about 10% of the patients with PH. PH2 is caused by dysfunction of the enzyme glyoxalate/hydroxypyruvate reductase (GRHPR) which occurs secondary to a mutation in the GRHPR gene located on chromosome 10.

Primary hyperoxaluria type 3 (PH 3) occurs in 10% PH cases. PH3 is caused by a genetic defect in the HOGA1 gene located on chromosome 9 which codes for the mitochondrial 4-hydroxy 2-oxoglutarate aldolase. This enzyme breaks down 4-hydroxy 2-oxoglutarate into pyruvate and glyoxalate which in turn is converted into oxalate.

In embodiments, the primary hyperoxaluria may be primary hyperoxaluria type 1, type2, or type3.

The term “secondary hyperoxaluria” refers to a disease or condition in which excess oxalate is absorbed into the gastrointestinal (GI) tract and then excreted in the urine.

The reasons for increased absorption can be due to (1) eating foods that contain high levels of oxalate or excess intake of oxalate precursors (i.e., dietary hyperoxaluria), (2) alterations in intestinal microflora or having medical conditions that cause the GI tract to absorb more oxalate such as Crohn's disease, inflammatory bowel disease, gastric bypass and other disorders in which nutrients are not properly absorbed (i.e., enteric hyperoxaluria), or (3) unknown reasons (i.e. idiopathic hyperoxaluria). Without being bound to theory, malabsorption, in the context of point (2) and (3), may induce increased free fatty acids (FFA) concentration in the gut which by binding calcium, results in less available free calcium to bind to oxalate. Secondly, increased concentrations of bile acids induce increase of permeability of the intestinal mucosa. Both mechanisms results in higher concentration of free oxalate, hence increasing gastro-intestinal absorption of oxalate.

In embodiments, hyperoxaluria may be determined by one or more of a urine test to measure levels of oxalate and optionally other specific enzymes; a blood test to measure the amount of oxalate in blood; and a scan (such as X-rays, ultrasound, and/or CT) of the kidneys and urinary tract to check for kidney stones or calcium oxalate crystals. Urine may also be checked for crystals.

Hyperoxaluria may be assessed by measuring urinary oxalate excretion in 24 h urine collection and adjustment of the oxalate excretion per 1.73 m² of the body surface area (e.g., expressed in mmol/1.73 m²/24 hours). Urinary oxalate excretion may also be measured per volume of urine (e.g., expressed in mg/L). Urinary oxalate/urinary creatinine ratios (e.g. mg oxalate/g creatinine) may be measured to determine the quality of the 24 h urine collection. The urinary creatinine is rather stable over time (24 hours), hence for instance false low urine collection can be detected by too low values of creatinine. The values can be corrected or the result can be rejected (Kamińska et al., 2020, Crit Rev Clin Lab Sci, 57, 5, 345-364). Urinary phosphate/urinary creatinine ratios (e.g. mg phosphate/g creatinine), urinary phosphate (e.g., phosphate mg/L), urinary calcium/urinary creatinine ratios (e.g. mg calcium/g creatinine) and/or urinary calcium (e.g., calcium mg/L) may as well be determined.

In a urinalysis, the amount of urinary oxalate, urinary calcium, or urinary phosphate can be measured in urine collected during 24 hours (h) by a patient (e.g., during a control period, during treatment, and/or after treatment). Urinary oxalate, urinary calcium, or urinary phosphate excretion may be expressed in mmol/24 h/1.73 m² or mg/24 h. Preferably, the excretion of urinary oxalate, urinary calcium, or urinary phosphate is expressed in mmol/24 h/1.73 m² per liter (L) of urine (referred to herein as “mmol/L”) or in mg/24 h/1.73 m² per liter of urine (referred to herein as “mg/L”) to take into account to the amount of excreted urine by the patient. It is important to take into account the volume of urine of the patient in 24 hours as hyperoxaluria patients are used to drink and urinate a lot. A mention of the excretion of urinary oxalate, urinary calcium, or urinary phosphate without taking into account the volume, e.g., in mmol/24 h/1.73 m², may lead to incorrect diagnosis. For instance, a subject having an excretion of 80 mg oxalate per 24 hours in 2 L of urine may not be diagnosed as having hyperoxaluria (i.e., 40 mg/L is below upper limit of normal (ULN) of 44 mg/L), while a subject having an excretion of 80 mg oxalate in per 24 hours in 1 L of urine will be diagnosed as having hyperoxaluria (i.e., 80 mg/L above ULN of 44 mg/L).

Expected urinary oxalate excretions in normal and diseased human patients are provided in Table 1.

TABLE 1
Expected human urinary oxalate excretion
in various states (as used in the art)

| | In mmol/24 h/ | In mg/24 h/ |
|---|---|---|
| Urinary oxalate excretion rates | 1.73 m² | 1.73 m² |
| Normal population: upper limit of | <0.50 | <44 |
| normal (ULN) | | |
| Dietary and idiopathic hyperoxaluria | 0.50-0.70 | 44-61 |
| Enteric hyperoxaluria | 0.60-1.0 | 53-87 |
| Primary hyperoxaluria | >1.0 | >87 to 250 |

As used herein, normal urinary oxalate excretion is defined as less than 0.50 mmol/L or less than 44 mg/L.

Hyperoxaluria may be characterized by urinary oxalate excretion of equal to or more than 0.50 mmol/L or 44 mg/L.

Primary hyperoxaluria may be characterized by urinary oxalate excretion of more than 1.0 mmol/L or more than 87 mg/L such as by urinary oxalate excretion of 88 mg/L to 250 mg/L.

Secondary hyperoxaluria may be characterized by urinary oxalate excretion of equal to or more than 0.50 mmol/L or 44 mg/L. Dietary and idiopathic hyperoxaluria may be characterized by urinary oxalate excretion of 0.50-0.70 mmol/L or 44-61 mg/L. Enteric hyperoxaluria may be characterized by urinary oxalate excretion of 0.60-1.0 mmol/L or 53-87 mg/L.

Diagnosis of PH and type may be provided by testing of AGXT, GRHPR and HOGA1 genes. There are 150 known mutations for AGXT, 16 for GRHP, and 15 for HOGA1. Williams et al. (2007, Clin Chem, 53: 1216-1221) showed that targeted analysis of the three most common mutations in AGXT (c.33_34insC, c.508G>A, and c.731T>C) provides the diagnosis in 34.5% PH1 patients while exon sequencing of exon 1, 4 and 7 increases the yield and allows diagnosis in 50% PH1 patients.

The term “oxaluria” refers to the excretion of oxalate in the urine or urinary oxalate.

The term “calciuria” refers to the excretion of calcium in the urine or urinary calcium.

An abnormally high amount of urinary calcium is called hypercalciuria and an abnormally low amount is called hypocalciuria.

The term “phosphaturia” refers to excretion of phosphates in the urine or urinary phosphate.

An abnormally high amount of urinary phosphate is called hyperphosphaturia and an abnormally low amount is called hypophosphaturia.

A further aspect relates to the pharmaceutical dosage form as defined herein, for use in the treatment of one or more of nephrolithiasis, nephrocalcinosis, or oxalosis in a subject, in particular a human subject. Accordingly, a further aspect provides an orally administrable pharmaceutical dosage form configured for targeted delivery to the distal part of the gastrointestinal tract of a subject, wherein the pharmaceutical dosage form comprises lanthanum or a pharmaceutically acceptable salt or oxide thereof, for use in the treatment of one or more of nephrolithiasis, nephrocalcinosis, or oxalosis in the subject, in particular the human subject. Preferably, a further aspect provides an orally administrable pharmaceutical dosage form comprising lanthanum or a pharmaceutically acceptable salt or oxide thereof, wherein the pharmaceutical dosage form is configured for targeted delivery of the lanthanum or pharmaceutically acceptable salt or oxide thereof to one or more of the ileum, cecum, or colon of a subject, in particular a human subject, for use in the treatment of one or more of nephrolithiasis, nephrocalcinosis, or oxalosis in the subject, in particular the human subject.

The terms “nephrolithiasis”, kidney stone disease”, or “urolithiasis” refer to a crystallopathy where a solid piece of material (e.g., kidney stone) develops in the urinary tract. Diagnosis of kidney stones is made on the basis of information obtained from the history, physical examination, urinalysis, and radiographic studies. Clinical diagnosis is usually made on the basis of the location and severity of the pain, which is typically colicky in nature (comes and goes in spasmodic waves). Pain in the back occurs when calculi produce an obstruction in the kidney.

In embodiments, the nephrolithiasis may be caused by or be related to calcium oxalate such as calcium oxalate stones or crystals.

The terms “nephrocalcinosis” or “Albright's calcinosis” refer to renal parenchymal calcification as diagnosed by radiology. The calcification may be diffuse, fine calcification. The term “nephrocalcinosis” is used to describe the deposition of both calcium oxalate and calcium phosphate. Nephrocalcinosis may cause acute kidney injury. Nephrocalcinosis may be diagnosed by imaging techniques including ultrasound (US), abdominal plain film and computer tomography (CT) imaging.

In embodiments, the nephrocalcinosis may be caused by or be related to calcium oxalate such as calcium oxalate deposition.

The term “oxalosis” refers to a disease or condition in which excess oxalate builds up in the extrarenal organs after the kidneys fail in subjects who have hyperoxaluria. This can lead to oxalate deposits in blood vessels, bones, and body organs.

In embodiments, the oxalosis may be caused by or be related to calcium oxalate such as calcium oxalate deposition, stones, or crystals.

In embodiments, the nephrolithiasis, nephrocalcinosis, or oxalosis in a subject may be caused by or related to calcium oxalate such as calcium oxalate deposition, stones, or crystals in the subject.

Related aspects provide:
a method of treating one or more of nephrolithiasis, nephrocalcinosis, or oxalosis in a subject in need of such a treatment, comprising administering, preferably orally administering, a therapeutically effective amount of a pharmaceutical dosage form as taught herein to the subject.
a method of treating one or more of nephrolithiasis, nephrocalcinosis, or oxalosis in a human subject in need of such a treatment, comprising administering, preferably orally administering, a therapeutically effective amount of a pharmaceutical dosage form as taught herein to the subject.
the use of a pharmaceutical dosage form as taught herein for the manufacture of a medicament for the treatment of one or more of nephrolithiasis, nephrocalcinosis, or oxalosis in a subject.
the use of a pharmaceutical dosage form as taught herein for the manufacture of a medicament for the treatment of one or more of nephrolithiasis, nephrocalcinosis, or oxalosis in a human subject.

The terms “subject”, “individual” or “patient” can be used interchangeably herein, and typically and preferably denote humans, but may also encompass reference to non-human animals, preferably warm-blooded animals, even more preferably mammals, such as, e.g., non-human primates, rodents, canines, felines, equines, ovines, porcines, and the like. The term “non-human animals” includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In embodiments, the subject is a non-human mammal. In embodiments, the subject is a human subject. The term does not denote a particular age or sex. Thus, adult and new-born subjects, as well as fetuses, whether male or female, are intended to be covered. Examples of subjects include humans, dogs, cats, cows, goats, and mice. The term subject is further intended to include transgenic species.

Suitable subjects may include without limitation subjects presenting to a physician for a screening for one or more of hyperoxaluria, nephrolithiasis, nephrocalcinosis, or oxalosis, subjects presenting to a physician with symptoms and signs indicative of one or more of hyperoxaluria, nephrolithiasis, nephrocalcinosis, or oxalosis, subjects diagnosed with one or more of hyperoxaluria, nephrolithiasis, nephrocalcinosis, or oxalosis, and subjects who have received an alternative (unsuccessful) treatment for one or more of hyperoxaluria, nephrolithiasis, nephrocalcinosis, or oxalosis.

In embodiments of the uses or methods as taught herein, the subject is a human. As shown in the examples, the present inventors found the optimal site in the gut of human subjects where release of lanthanum should take place and chelation of oxalate should start to get the combined effect of reduction of hyperoxaluria without phosphate depletion, namely in the distal part of the gastrointestinal tract of human subjects, in particular in one or more of the ileum, cecum or colon of human subjects.

In embodiments of the uses or methods as taught herein, the subject may be a human subject having primary hyperoxaluria and treated with lumasiran, e.g. for at least 6 months, e.g., for at least 8 months, at least 10 months, or at least 1 year, and/or according to EMA lumasiran treatment schedule, i.e., starting with 4 doses with an interval of 1 month followed by 1 dose every 3 months in adults. In embodiments, the subject may be a human subject having primary hyperoxaluria, treated with lumasiran, and presenting a suboptimal result with lumasiran alone. A suboptimal result with lumasiran alone is obtained when the primary end point for lumasiran treatment, namely a 24 -hour urinary oxalate excretion lower than about 1.5 times the upper limit of the normal (ULN), namely 68 mg/24 hrs, is not reached. Treatment with the pharmaceutical dosage form as taught herein in combination with lumasiran may advantageously allow to decrease the urinary oxalate below the ULN of 44 mg/L.

The term “lumasiran”, having trade name Oxlumo (Alnylam Pharmaceuticals Inc.), refers to a double-stranded siRNA for the treatment of primary hyperoxaluria that reduces levels of glycolate oxidase (GO) enzyme by targeting the HAO1 messenger ribonucleic acid (mRNA) in hepatocytes through RNA interference. Decreased GO enzyme levels reduce the amount of available glyoxylate, a substrate for oxalate production.

In embodiments of the uses or methods as taught herein, the subject may be a human subject having primary hyperoxaluria and having been diagnosed with impaired renal function, and optionally being in need of kidney transplantation. Treatment with the pharmaceutical dosage form as taught herein in combination with lumasiran and frequent dialysis, e.g. over a period of at least 2-4 months, may advantageously allow to decrease the oxalate load in the body of such subjects.

In embodiments of the uses or methods as taught herein, the subject is a human subject having primary or secondary hyperoxaluria and impaired renal function. In embodiments of the uses or methods as taught herein, the subject is a human subject having primary or secondary hyperoxaluria and renal failure.

For instance, the subject may be a human subject having primary hyperoxaluria and chronic kidney disease 3-5 (CKD3-5). Treatment with the pharmaceutical dosage form as taught herein may advantageously allow to solubilize and remove most of the calcium oxalate from the kidneys through secretion from the serum to the ileum and colon.

In embodiments of the uses or methods as taught herein, the subject is a human subject having primary hyperoxaluria and terminal CKD or impaired renal function (eGFR<40 ml/min). These subjects may be on dialysis and in need of kidney and liver transplant. Lumasiran can largely suppress liver production of oxalate, thereby avoiding liver transplantation. Treatment with the pharmaceutical dosage form as taught herein with its high capacity of chelation of secreted oxalate and elimination via the intestine in combination with lumasiran may advantageously allow to decrease the oxalate load in the body of such subjects. For the avoidance of doubt, the kidney to be transplanted must be placed in an environment of oxalate concentrations below 44 mg/L or 0.51 mmol/L in the urine in order not to show crystal formation after the kidney transplant when urine formation starts again.

In embodiments of the uses or methods as taught herein, the subject is a human subject having secondary hyperoxaluria and moderate renal insufficiency.

In embodiments of the uses or methods as taught herein, the pharmaceutical dosage form may be administered:
once or twice a day;
in an amount of from 100 mg to 2000 mg lanthanum per administration;
in an amount of from 100 mg to 4000 mg lanthanum per day; and/or
for a period of at least one month; such as for a period of at least two months.

Preferably, the pharmaceutical dosage form is orally administered.

In embodiments of the uses or methods as taught herein, the pharmaceutical dosage form may be administered at least once a day such as at least twice a day, at least three times a day or at least four times a day. Preferably, the pharmaceutical dosage form is administered once a day or twice a day, thereby improving patients' compliance. In embodiments, the pharmaceutical dosage form may be administered once a day such as in the morning or in the evening. In embodiments, the pharmaceutical dosage form may be administered twice a day such as in the morning and in the evening. Preferably, the pharmaceutical dosage form is administered immediately before (i.e., at most 60 min, at most 30 min, at most 15 min, or at most 5 min before), during, or immediately after (i.e., at most 60 min, at most 30 min, at most 15 min, or at most 5 min after) a meal, such as immediately before, during, or immediately after the most copious meal(s) of the day.

In embodiments of the uses or methods as taught herein, the pharmaceutical dosage form may be administered in an amount of from 100 mg to 2000 mg lanthanum per administration. In embodiments, the pharmaceutical dosage form may be administered in an amount of from 250 mg to 1500 mg, from 500 mg to 1000 mg, or from 750 mg to 1000 mg lanthanum per administration. Per administration, at least one dosage form (e.g., capsule) may be administered, such as at least two dosage forms, at least three dosage forms, at least four dosage forms, at least five dosage forms, or at least six dosage forms.

In embodiments of the uses or methods as taught herein, the pharmaceutical dosage form may be administered in an amount of from 100 mg to 4000 mg lanthanum per day. In embodiments, the pharmaceutical dosage form may be administered in an amount of from 200 mg to 4000 mg, from 500 mg to 3000 mg, from 1000 mg to 2000 mg, or from 1500 mg to 2000 mg lanthanum per day.

In embodiments of the uses or methods as taught herein, the pharmaceutical dosage form may be administered for a period of at least one month. In embodiments, the pharmaceutical dosage form may be administered for a period of at least two months, at least two and a halve months, at least three months, at least four months, at least five months, or at least six months. In embodiments, the pharmaceutical dosage form may be administered for a period of at least seven months, at least eight months, at least nine months, at least ten months, at least eleven months, or longer, such as for a period of at least one year, at least two years, at least three years, at least four years, at least five years, or longer.

The pharmaceutical dosage form as taught herein does not lead to phosphate depletion, thereby advantageously allowing long term treatment of primary and secondary hyperoxaluria patients.

The pharmaceutical dosage form as taught herein allows treatment of one or more of hyperoxaluria, nephrolithiasis, nephrocalcinosis, or oxalosis.

As used herein, a phrase such as “a subject in need of treatment” includes subjects that would benefit from treatment of a given condition, particularly one or more of hyperoxaluria, nephrolithiasis, nephrocalcinosis, or oxalosis. Such subjects may include, without limitation, those that have been diagnosed with said condition, those prone to develop said condition and/or those in who said condition is to be prevented.

The terms “treat” or “treatment” encompass both the therapeutic treatment of an already developed disease or condition, such as the therapy of an already developed hyperoxaluria, nephrolithiasis, nephrocalcinosis, or oxalosis, as well as prophylactic or preventive measures, wherein the aim is to prevent or lessen the chances of incidence of an undesired affliction, such as to prevent occurrence, development and progression of hyperoxaluria, nephrolithiasis, nephrocalcinosis, or oxalosis. Beneficial or desired clinical results may include, without limitation, alleviation of one or more symptoms or one or more biological markers, diminishment of extent of disease, stabilized (i.e., not worsening) state of disease, delay or slowing of disease progression, amelioration of the disease state, and the like. The term may encompass ex vivo or in vivo treatments.

The term “prophylactically effective amount” refers to an amount of a pharmaceutical dosage form or an active compound such as lanthanum or pharmaceutically acceptable salt or oxide thereof that inhibits or delays in a subject the onset of a disorder as being sought by a researcher, veterinarian, medical doctor, or other clinician.

The methods as taught herein allow to administer a therapeutically effective amount of a pharmaceutical dosage form or an active compound such as lanthanum or pharmaceutically acceptable salt or oxide thereof in subjects having one or more of hyperoxaluria, nephrolithiasis, nephrocalcinosis, or oxalosis. The term “therapeutically effective amount” as used herein, refers to an amount of active compound or pharmaceutical dosage form that elicits the biological or medicinal response in a subject that is being sought by a surgeon, researcher, veterinarian, medical doctor, or other clinician, which may include, without limitation, alleviation of the symptoms of the disease or condition being treated. Methods are known in the art for determining therapeutically effective doses of lanthanum or pharmaceutically acceptable salts thereof.

Appropriate therapeutically effective doses of an active compound as taught herein may be determined by a qualified physician with due regard to the nature of the active compound, the disease condition and severity, and the age, size, and condition of the patient.

A further aspect provides a method for preparing a pharmaceutical dosage form as defined herein, in particular a capsule, the method comprising:
filling a capsule with lanthanum or a pharmaceutically acceptable salt or oxide thereof;
closing the capsule; and
dipping the capsule in a coating solution configured for targeted delivery to the distal part of the gastrointestinal tract of a subject, in particular to the ileum, cecum, and/or colon of a subject.

In embodiments, the method may comprise the prior step of opening the capsule.

In embodiments of the methods as taught herein:
the capsule may be dipped at least three times in the coating solution;
the dipping time in the coating solution may be at least 10 seconds; and/or
the coating may be dried prior to a next dipping step, such as wherein the coating may be dried for at least 3 minutes prior to a next dipping step.

In embodiments, the coating solution may comprise one or more materials configured for targeted delivery to the distal part of the gastrointestinal tract of a subject, in particular to the ileum, cecum, and/or colon of a subject. In embodiments, the coating solution may comprise a pH sensitive polymer, a biodegradable polymer, a polysaccharide, or a combination thereof. In embodiments, the coating solution may comprise a polymer configured for targeted delivery to the distal part of the gastrointestinal tract of a subject, as taught herein, in particular to the ileum, cecum, and/or colon of a subject.

In embodiments, the coating solution may further comprise one or more additives or auxiliary agents such as a solvent, a plasticizer, a carrier, a surfactant, a thickener, a buffering agent, an antioxidant, a preservative, an aroma, or a colorant. Suitable auxiliary agents are known in the art.

The solvent may be an organic solvent such as acetone. The solvent may be an aqueous solvent (i.e. aqueous carrier or aqueous solution). In embodiments, the coating solution may not contain an organic solvent. In embodiments, the coating may not contain an organic solvent.

The plasticizer may be any suitable plasticizer known in the art such as triethyl citrate.

The term “plasticizer” refers to a substance or composition that produces or promotes plasticity or viscosity of a material.

In embodiments, the coating solution may comprise a copolymer of methacrylic acid and methyl methacrylate, a solvent, and a plasticizer. For example, the coating solution may comprise a copolymer of methacrylic acid and methyl methacrylate in a ratio of 1:2, acetone, and triethyl citrate.

In embodiments, the coating solution may comprise from 2% to 20% by weight of a copolymer of methacrylic acid and methyl methacrylate, from 75% to 97.5% by weight of a solvent, and from 0.5% to 5% by weight of a plasticizer. For example, the coating solution may comprise 10% by weight of a copolymer of methacrylic acid and methyl methacrylate in a ratio of 1:2, 89% by weight of acetone, and 1% by weight of triethyl citrate.

The terms “% by weight”, “% (w/w)”, “percentage by mass”, “percentage by weight” or “wt %” may be used interchangeably herein and refer to the mass fraction wᵢ times 100. The mass fraction wᵢ is the ratio of the mass mᵢ of one compound to the mass mₜₒₜ of the total mixture (e.g., the coating solution as taught herein), as defined in (1):

$${w}_{i} = \frac{{m}_{i}}{{m}_{tot}} \qquad ( 1 )$$

In embodiments, the coating solution may be prepared by:
dissolving the plasticizer (e.g., triethyl citrate) in a solvent (e.g., acetone); and
adding a copolymer of methacrylic acid and methyl methacrylate (e.g., Eudragit S 100) while stirring until completely dissolved, thereby obtaining the coating solution.

Stirring may be performed with a magnetic stirrer. Stirring may be performed for a period of at least 30 min, such as for a period of at least 1 h or at least 2 h.

In embodiment of the methods as taught herein, the capsule may be dipped at least three times in the coating solution, such as at least four times, at least five time, or at least six times in the coating solution.

Preferably, the capsule is dipped four times in the coating solution.

In embodiment of the methods as taught herein, the capsule may be dipped in the coating solution (i.e., dipping and withdrawal time) for at least 10 seconds (sec), such as for at least 15 sec, at least 20 sec, at least 25 sec, at least 30 sec, or longer. Preferably, the capsule is dipped in the coating solution (i.e., dipping and withdrawal time) for 15 sec.

In embodiment of the methods as taught herein, the coating may be dried prior to a next dipping step for at least 3 minutes prior to a next dipping step. In embodiment of the methods as taught herein, the coating may be dried for at least 4 minutes (min), at least 5 min, at least 6 min, at least 7 min, at least 8 min, at least 9 min, at least 10 min, or at least 15 min prior to a next dipping step. Preferably, the coating is dried for 5 min prior to a next dipping step.

In embodiments, the coating may be dried for at least 5 minutes (min), at least 8 min, at least 10 min, at least 15 min, at least 20 min, at least 25 min, at least 30 min, or at least 45 min after the last dipping step.

Preferably, the coating is dried for 15 min after the last dipping step.

In embodiments, the pharmaceutical dosage form as taught herein such as the capsule may be stored at room temperature (e.g., 20-24° C.) until use.

The present application also provides aspects and embodiments as set forth in the following Statements:

Statement 1. An orally administrable pharmaceutical dosage form configured for targeted delivery to the distal part of the gastrointestinal tract of a subject, wherein the pharmaceutical dosage form comprises lanthanum or a pharmaceutically acceptable salt or oxide thereof.

Statement 2. The pharmaceutical dosage form according to statement 1, wherein the distal part of the gastrointestinal tract may be the ileum, the cecum, the colon, or a combination thereof.

Statement 3. The pharmaceutical dosage form according to statement 1 or 2, which is a solid or semisolid dosage form.

Statement 4. The pharmaceutical dosage form according to any one of statements 1 to 3, which is a capsule or a tablet, such as a capsule filled with a composition comprising the lanthanum or pharmaceutically acceptable salt or oxide thereof; preferably wherein the lanthanum or the pharmaceutically acceptable salt or oxide thereof is in powder form.

Statement 5. The pharmaceutical dosage form according to any one of statements 1 to 4, wherein the pharmaceutical dosage form or capsule comprises a coating configured for targeted delivery to the distal part of the gastrointestinal tract of a subject; preferably wherein the coating comprises a pH sensitive polymer, a biodegradable polymer, a polysaccharide, or a combination thereof.

Statement 6. The pharmaceutical dosage form according to any one of statements 1 to 5, wherein the pharmaceutical dosage form or the coating comprises a copolymer of methacrylic acid and C₁₋₄alkylester of methacrylic acid; preferably a copolymer of methacrylic acid and methyl methacrylate; more preferably a copolymer of methacrylic acid and methyl methacrylate in a ratio of 1:2.

Statement 7. The pharmaceutical dosage form according to any one of statements 4 to 6, wherein the capsule is made of one or more materials selected from the group consisting of cellulose acetate phthalate (CAP), hydroxypropyl methylcellulose (HPMC), gelatin, pullulan, starch, a water-soluble polyvinyl derivative, a polyethylene glycol, and a copolymer of C₁₋₄alkylester of (meth)acrylic acid and (meth)acrylic acid.

Statement 8. The pharmaceutical dosage form according to any one of statements 1 to 7, wherein the pharmaceutical dosage form or the capsule comprises at least three layers of the coating, preferably wherein the capsule comprises at least four layers of the coating; and/or wherein the thickness of the coating is at least 20.0 μm, such as at least 25.0 μm, at least 30.0 μm, or at least 35.0 μm.

Statement 9. The pharmaceutical dosage form according to any one of statements 1 to 8, wherein the pharmaceutically acceptable salt or oxide of lanthanum is lanthanum carbonate or lanthanum oxide; preferably wherein the pharmaceutical dosage form comprises lanthanum carbonate.

Statement 10. The pharmaceutical dosage form according to any one of statements 1 to 9, wherein the pharmaceutical dosage form comprises 100 mg to 4000 mg of elemental lanthanum.

Statement 11. The pharmaceutical dosage form according to any one of statements 1 to 10, wherein the pharmaceutical dosage form is a capsule filled with a composition comprising the lanthanum or pharmaceutically acceptable salt or oxide thereof, and coated with a copolymer of methacrylic acid and C₁₋₄alkylester of methacrylic acid.

Statement 12. The pharmaceutical dosage form according to any one of statements 1 to 11, for use in the treatment of hyperoxaluria in a subject, optionally wherein the hyperoxaluria is primary hyperoxaluria or secondary hyperoxaluria.

Statement 13. The pharmaceutical dosage form according to any one of statements 1 to 11, for use in the treatment of one or more of nephrolithiasis, nephrocalcinosis, or oxalosis in a subject.

Statement 14. The pharmaceutical dosage form for use according to any one of statements 12 or 13, wherein the subject is a human.

Statement 15. The pharmaceutical dosage form for use according to any one of statements 12 to 14, wherein the pharmaceutical dosage form is administered:
once or twice a day;
in an amount of from 100 mg to 2000 mg lanthanum per administration;
in an amount of from 100 mg to 4000 mg lanthanum per day; and/or
for a period of at least one month; such as for a period of at least two months.

The above aspects and embodiments are further supported by the following non-limiting examples.

### EXAMPLES

### Example 1: Preparation of an Orally Administrable Pharmaceutical Dosage Form Configured for Targeted Delivery to the Distal Part of the Gastrointestinal Tract of a Subject According to an Embodiment of the Invention

This example provides the process for the preparation of an orally administrable pharmaceutical dosage form configured for targeted delivery to the distal part of the gastrointestinal tract of a subject according to an embodiment of the invention, in particular a capsule comprises a coating configured for targeted delivery to the distal part of the gastrointestinal tract of a subject.

The capsules were cellulose acetate phthalate (CAP) capsules. As active substance, the preparation started from Fosrenol sachets (Shire Pharmaceutics, Dublin, Ireland) containing 750 mg of elemental lanthanum under the form of lanthanum carbonate (La₂(CO₃)₃). The other compounds of Fosrenol were dextranes (hydrated), colloidal water-free silica, and magnesium stearate.

Capsules were filled with the powder blend from the “Fosrenol sachet 750”. One third (⅓) of the sachet was filled in a capsule, meaning that 250 mg elemental lanthanum was used, corresponding to ±500 mg of lanthanum carbonate per capsule.

The capsules were coated. The composition of the coating solution was:
Eudragit S 100: 10% (w/w) (Evonik Industries, Belgium)
triethyl citrate: 1% (w/w) (Sigma Aldrich, Germany)
acetone: 89% (w/w) (Acetone extra pure, Fagron, The Netherlands)

The coating solution was prepared by dissolving triethyl citrate (plasticizer) in acetone. Eudragit S 100 was gradually added while stirring on a magnetic stirrer until completely dissolved, which took between 1-2 hours.

Filled capsules, size 00, were manually coated with Eudragit using a manual coater (Feton International) according to the following steps: a first dipping of the body of the capsules, dipping and withdrawal time was 15 seconds; repeat dipping of the body of the capsule, up to 3 times. Finally, at the end of our testing (see below), the pharmaceutical dosage form illustrating the invention was prepared by performing a 4^{th} coating step of the body with a coating configured for targeted delivery to the distal part of the gastrointestinal tract of a subject.

Drying time between the layers was 5 minutes and the 4^{th} layer was let for drying during 15 minutes.

Prepared capsules were stored at room temperature between 20-24° C.

Using a manual screw micrometer, the mean value of thickness of the uncoated capsules was 0.145 mm±0.0054 mm (n=5). The mean value of thickness of the coated capsules (4 layers) was 0.174 mm±0.0037 mm (n=5).

Testing the efficiency of the coating was performed using two different milieus, an acidic one and an more basic one.

In the first test, capsules were coated with three layers as described in the protocol. One coated capsule was placed in a measuring cup with acetate buffer pH 4.6 (acidity of gastric juice during digestion).The temperature was maintained at 35° C. and placed on the magnetic stirrer. After 2 h the capsule was not intact.

In the second test, capsules were coated with three layers as described in the protocol. Coated capsules were placed in a carbonate buffer of pH 8. A colour change appeared at the seam after 15 minutes about half way through the capsule and the coating began to soak and dissolve. 20 min later the content was released in the surrounding liquid.

Pilot experiments were performed using capsules with different coating procedures (none, 1, 2, 4 and 5 layers) in a normal volunteer. Daily 24 h collections of urine were performed during the time of the experiment: control (i.e., before treatment), during treatment, during wash out periods (i.e., after treatment). In all these 24 h urinary collections, the oxalate, phosphate, calcium, and creatinine was determined. Determination of creatinine allowed to confirm the correctness of the 24 h collections. Using the capsules without coating or with one or two coating layers, there was no or negligible effect on the oxaluria, phosphaturia and calciuria (results not shown).

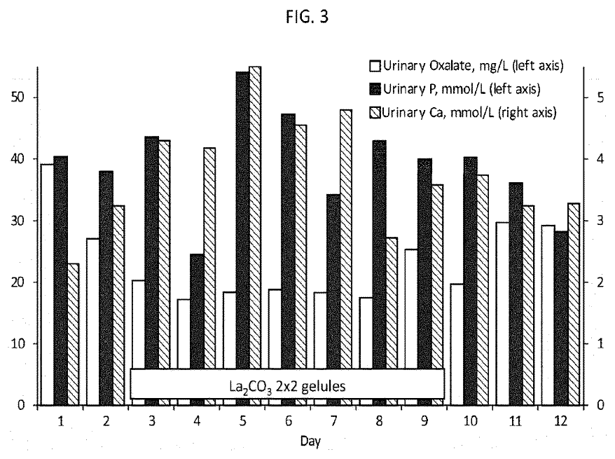

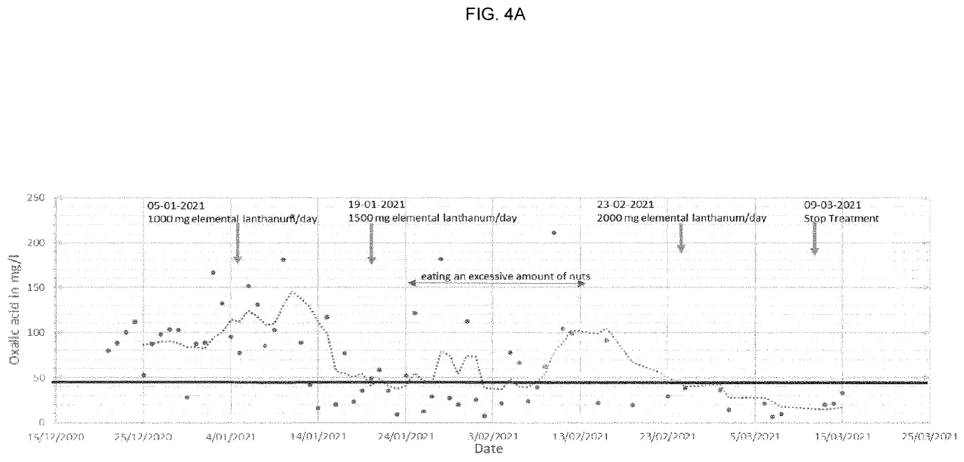

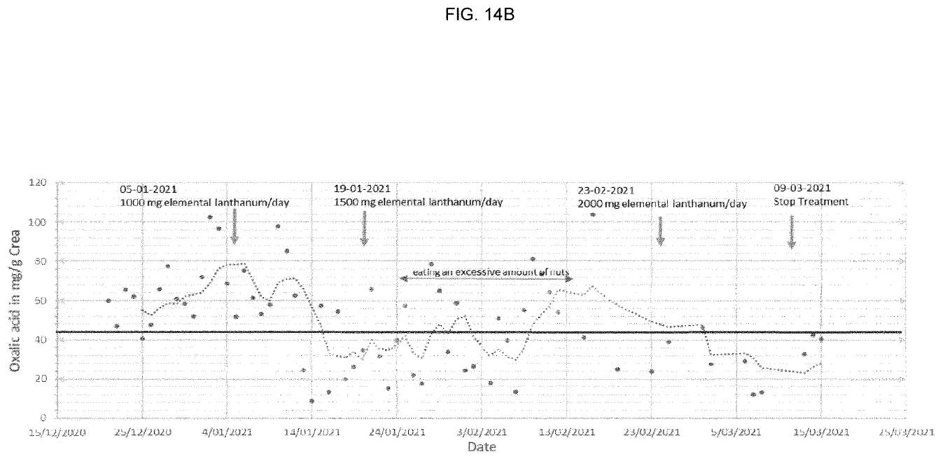

However, when the four times coated capsules were used, there was a clear effect on the oxaluria, up to more than 40% decrease in urinary oxalate compared to the control values (see Example 2, FIG. 3). No clear effect was seen on the phosphaturia and calciuria during this short clinical test (see Example 2, FIG. 3). Two days after arrest of intake, oxaluria was comparable to the control values before the start of the experiment (see Example 2, FIG. 3). When using capsules with 5 layers of the coating, this seemed not to increase the effect on the oxalate chelation in patients above the use of capsules with 4 layers of the coating.

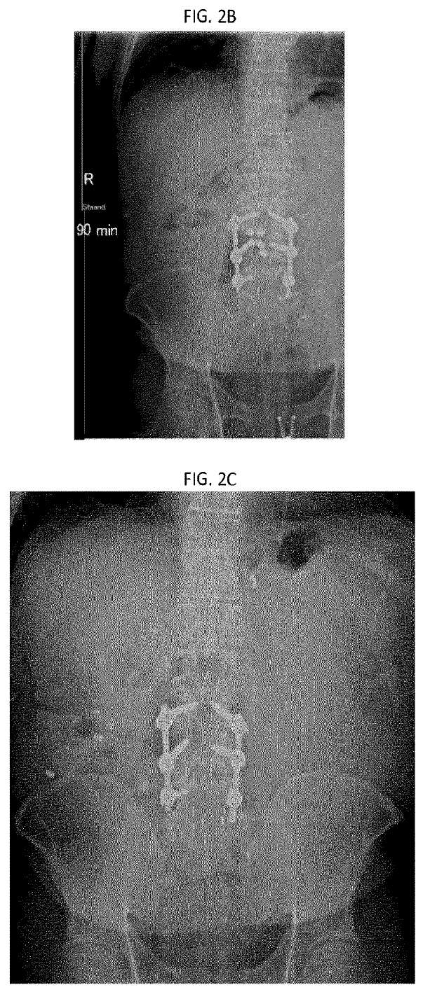

After radiographic monitoring of the behaviour of the uncoated capsule in the intestinal tract of a test subject, a rapid release of the capsule was observed at the proximal parts of the gastrointestinal tract (results not shown). Therefore, capsules were coated with a 4^{th} layer and optionally 5^{th} layer of the coating with an acceptable result of targeted release (see Example 2, FIG. 2 for capsules comprising 4 layers of coating).

The pharmaceutical dosage form illustrating the invention, in particular capsules illustrating the invention, hence contained 250 mg elemental lanthanum and were coated with four layers of a coating configured for targeted delivery to the distal part of the gastrointestinal tract of a subject, in particular to one or more of the ileum, cecum, or colon of a subject.

Further methods for the preparation of pharmaceutical dosage forms illustrating the invention involved the use of capsules having size 00, size 0, size 1, and size 2. These capsules were filled with lanthanum to contain 560 mg, 420 mg, 295 mg, and 230 mg of elemental lanthanum, respectively.

### Example 2: Release of Lanthanum Carbonate in the Distal Part of the Gastrointestinal Tract from a Pharmaceutical Dosage Form According to an Embodiment of the Invention

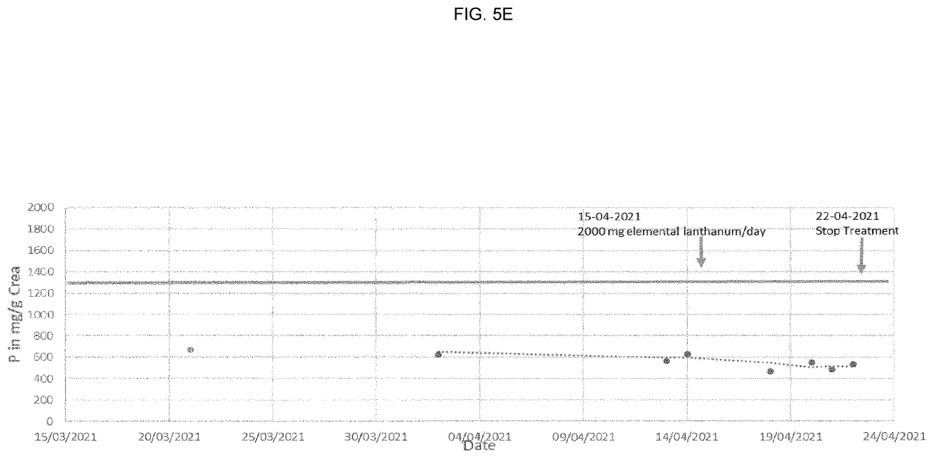

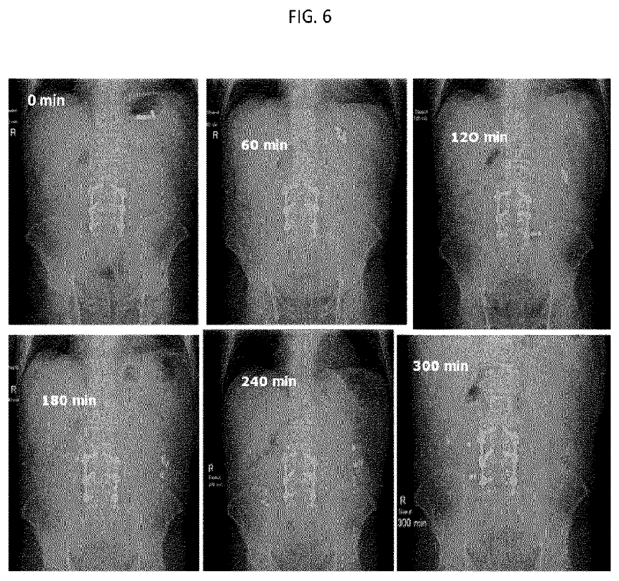

Using a Shimadzu Sonialvision G4 radiography device, a intestinal transit study of the capsules illustrating the invention (i.e. four times coated according to Example 1) with X-rays was performed. Serial images in standing anteroposterior direction of the abdomen of a normal volunteer were taken after oral ingestion of two capsules (of 250 mg elemental lanthanum each). Images were taken every 30 minutes for 360 min (6 hours). Immediately after swallowing both capsules, the capsules were visible in the stomach at “minute 0” on the first shot (FIG. 6). After 30 minutes, the capsules were breaking up into four larger fragments that moved up into the duodenum and jejunum (FIG. 2A). After 60 minutes and 90 minutes, the larger fragments moved further distally (FIG. 2B, FIG. 6). 120 minutes after oral ingestion, the larger capsular fragments were approximately at the level of the distal ileum and clearly visible punctiform lanthanum particles were already visible (FIG. 6). From 150 minutes, the larger capsules and the punctiform particles reached the level of the caecum via Bauhin's valve (results not shown). Over the next 30 min series, the larger capsular lanthanum fragments gradually diminished in number and the punctiform particles progressively increased, indicating further release of the capsules (FIG. 6). After 240 min, the lanthanum was released in the colon (FIG. 2C, FIG. 6). On the last exposure at 360 minutes, all debris was in the colon and the most distal capsule residue was in the hepatic corner of the colon.

This investigation was also performed in two other patients with primary hyperoxaluria and showed the same results.

Further, the effect of the oral intake of two capsules illustrating the invention (250 mg of lanthanum each; 4 times coated capsules) in the morning and two in the evening (250 mg of lanthanum each) (i.e., total daily dose of 1000 mg elemental lanthanum) was investigated during seven days in a normal human individual (day 3 to 9 in FIG. 3). The urinary levels, in the 24 h urine collections, of oxalate (mg/L), phosphate (P) (in mmol/L), and calcium (Ca) (in mmol/L) were determined (FIG. 3). FIG. 3 illustrates the effect of 1000 mg/day in four doses of 250 mg elemental lanthanum (2×250 mg morning, 2×250 mg evening) on the urinary oxalate (mg/L) elimination in a normal volunteer.

There was a clear effect on the oxaluria, with up to 46% decrease in urinary oxalate during 7 days of treatment compared to the control values (FIG. 3, black bars). No measurable effect was seen on the phosphaturia (dark grey) and calciuria (light grey) during this short clinical test in a normal individual after 7 days of treatment (FIG. 3). Two days after arrest of intake, oxaluria was comparable to the control values before the start of the experiment (FIG. 3, day 11).

These results and the X-ray results confirmed that the lanthanum was mainly released in the ileum and proximal colon.

### Example 3: Treatment of Patients with Primary and Secondary Hyperoxaluria Using a Pharmaceutical Dosage Form According to an Embodiment of the Invention

A proof-of-concept study was started in 4 patients with primary hyperoxaluria and 1 patient with secondary hyperoxaluria. The patients were elected from University Hospital Ghent (Belgium) (n=2) and University Hospital Antwerp (Belgium) (n=2) and corresponded to the inclusion/exclusion criteria: age: more than 9 years of age, genetically confirmed primary oxaluria, estimated glomerular filtration rate (eGFR)>50 ml/min/1.73 m², not treated with Lumasiran (Oxlumo), urinary oxalate excretion of at least 0.60 mmol (52.2 mg) per 24 hours per 1.73 m² and no clinical evidence of extrarenal systemic oxalosis. The patients were asked to continue the primary hyperoxaluria standard-of-care regimen in place at the time of enrolment in this study.

The primary end points were defined as an:
oxaluria concentration of less than 0.50 mmol/L (44 mg/L) associated with a calciuria below 4 mmol/L (160 mg/L). These two conditions are considered as essential in the setting of absence of calcium oxalate crystal formation in the urine.
a stable normal concentration of urinary phosphate (>350 mg/L-1200 mg/L) and serum (30-45 mg/L), indicating absence of any form of development of a phosphate depletion over time.
a stable or slightly decreased calciuria indicating a calcium channel blocking effect of the treatment.

### Laboratory Investigations

The different parameters of the primary hyperoxaluria patients are summarized in Table 2. All laboratory analyses (haematological, liver tests and renal function tests) were performed in the Algemeen Medisch Laboratorium (A.M.L., Antwerp, Belgium) according to the international standards. Oxalate in urine and serum were determined with Liquid Chromatography-tandem-Mass Spectrometry (LC-tandem-MS). Oxalic acid-C13 was used as internal standard. The samples were hydrolysed and derivatized with a Hydrochloric Acid-Butanol mixture. After incubation and centrifugation, one microliter of the upper layer was injected into the instrument. Good to excellent results were obtained in the quality control of the “Referenzinstitut fur Bioanalytik” Bonn, Prof D.C. Knabbe, 2021.

TABLE 2
Primary hyperoxaluria patients (n = 4): Characteristics and effects of treatment with a pharmaceutical dosage form illustrating the invention

| | | | | | | | | | | Oxaluria | | |
|---|---|---|---|---|---|---|---|---|---|---|---|---|
| | | | | | | | | | | (mg/L) | | |
| | | | | | | | | | | **** | | |
| | | | | | | | | | Oxaluria | Start - - - | | |
| | | | Type of | | | End of | | | (mg/24 h) | End (% | Phospha- | |
| | | Year | primary | | Baseline | treatment | eGFR | Diuresis/ | start - - - | decrease) | turia | Calciuria |
| Patient | | of | hyper- | Nephro- | oxaluria | oxaluria | (ml/min/ | L/24 | end of | Treatment | (mg/L)** | (mg/L)*** |
| number | Sex | birth | oxaluria | lithiasis | mg/L* | mg/L* | 1.73 m²) | hours | treatment | duration | mean ± SD | mean ± SD |
| 1 | male | 1998 | 1 | ++ | 103 | 28 | 102 | 2.4 ± 0.32 | 308 - - - 38 | 94 - - - 18 | 641 ± 283 | 23 ± 10 |
| | | | | | | | | | | (81%) | Stable over | Stable |
| | | | | | | | | | | 2.5 months | time | over time |
| 2 | male | 2008 | 1 | ++ | 53 | 20 | 109 | 2.8 ± 0.16 | 72 - - - 43 | 54 - - - 24 | 306 ± 116 | 25 ± 12 |
| | | | | | | | | | | (56%) | Stable over | Stable |
| | | | | | | | | | | 3 months | time | over time |
| 3 | female | 2011 | 3 | + | 55 | 22 | 98 | 1.1 ± 0.35 | 75 - - - 20 | 49 - - - 23 | 911 ± 460 | 187 ± 79 |
| | | | | | | | | | | (53%) | Stable over | Stable |
| | | | | | | | | | | 2 months | time | over time |
| 4 | male | 1999 | 1 | +++ | 115 | 62 | 74 | 2.4 ± 0.6 | 245 - - - 94 | 165 - - - 28 | 447 ± 137 | 17 ± 11 |
| | | | | | | Still | | | | (83%) | Stable over | Stable |
| | | | | | | treated | | | | 1 year 7 | time | over time |
| | | | | | | | | | | months | | |

*Oxaluria normal value: less than 44 mg/L;
**Phosphaturia normal value: 350-1200 mg/L;
***Calciuria normal value: less than 160 mg/L;
**** Oxaluria (mg/L): average values calculated based on the results of the samples for which the creatinine control measurements were acceptable

### Dosages of the Pharmaceutical Dosage Form Illustrating the Invention

Patients received the pharmaceutical dosage form illustrating the invention as prepared in Example 1, i.e., capsule comprising 250 mg elemental lanthanum and four layers of coating configured for targeted delivery to the distal part of the gastrointestinal tract of a subject. At the start of the treatment, two capsules were given at the most copious meals of the day, i.e., two capsules in the morning and 2 capsules in the evening (i.e., 4×250 mg=1000 mg elemental lanthanum/day). Depending on the results of oxalate excretion in the urine (concentrations >44 mg/L, i.e. upper limit of normal), this starting dose was increased to 3 capsules in the morning and 3 capsules in the evening (i.e., 6×250 mg=1500 mg elemental lanthanum/day) and up to 4 capsules in the morning and 4 capsules in the evening (i.e., 8×250 mg=2000 mg elemental lanthanum/day) at the same timing as for the starting dose.

### Patients

From the beginning of this proof-of-concept study, patients were asked to collect 24 hours urine during the control period (i.e., before treatment), during the treatment period, and after arrest of the treatment. In all these 24 h collections, oxalate, phosphate, calcium, and creatinine were determined. Determination of creatinine allowed to confirm the correctness of the 24 h collections.

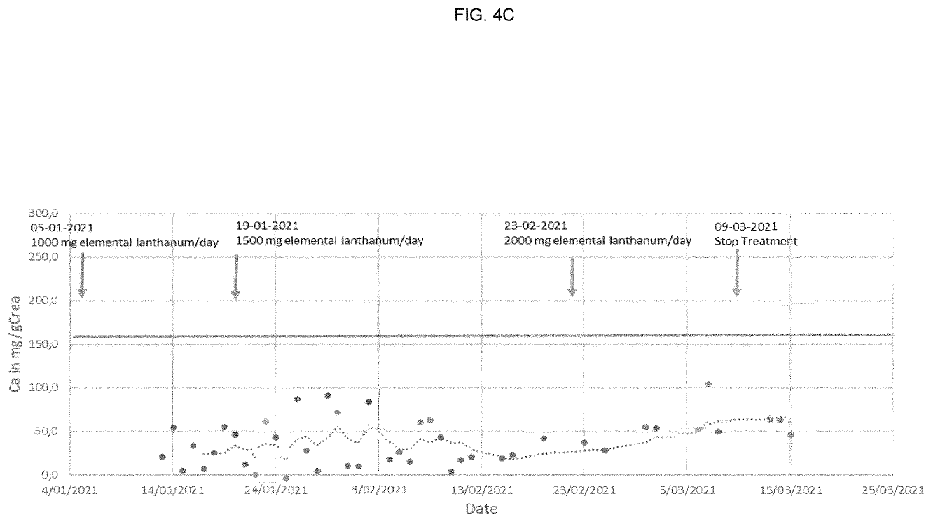

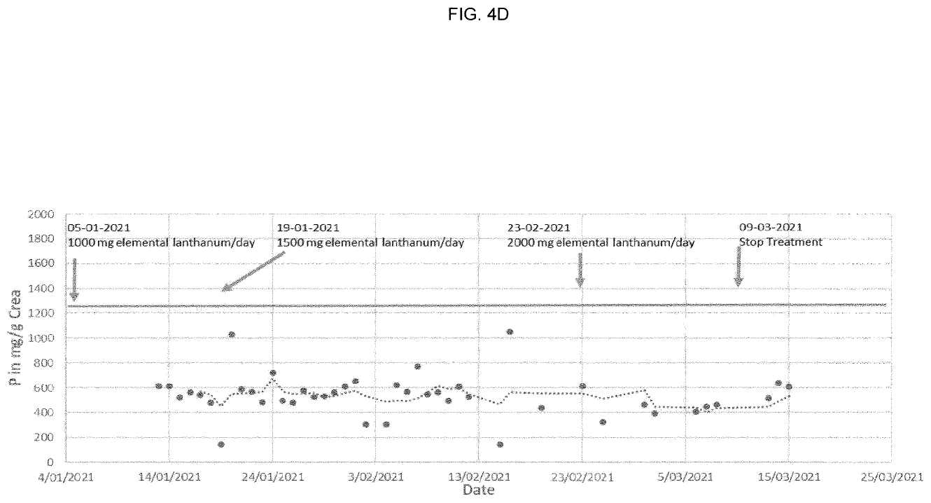

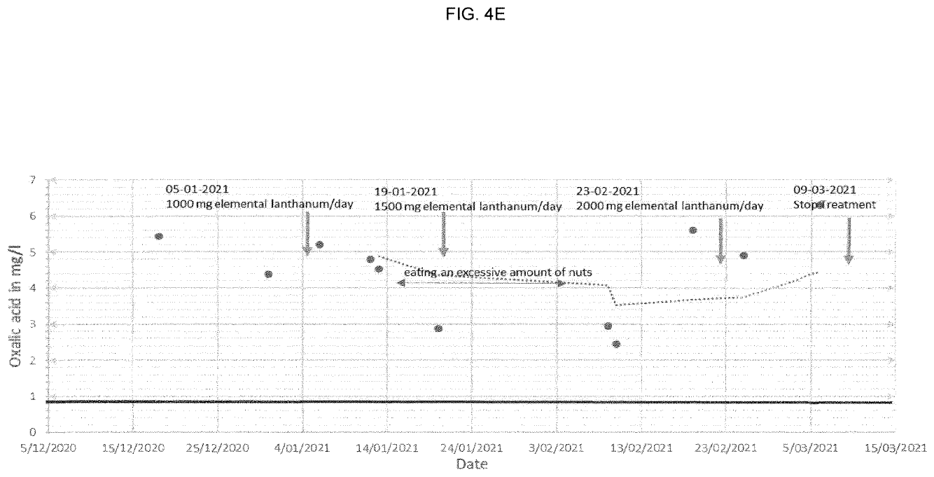

Three out of the four patients with primary hyperoxaluria (see Table 2) had a clear positive response after 2.5 to 3 months of the treatment. FIG. 4 demonstrates (A) the oxaluria (mg/L), (B) oxaluria (mg/gr creatinine), (C) calciuria (mg/gr creatinine), (D) phosphaturia (mg/gr creatinine) and (E) oxalate in the serum (mg/L) before, during and after treatment (2.5 month) with the pharmaceutical dosage form illustrating the invention in one of the patients with a serious form of type 1 primary hyperoxaluria (patient 1). As indicated in FIG. 4, the patient was eating nuts during part of the treatment for low phosphate. The therapeutic response was excellent as determined by the different ways in which the oxalate elimination was measured. The normal phosphaturia at the start of the treatment remained unchanged during the treatment time. This was also the case for the calciuria (FIG. 4).

The fourth primary hyperoxaluria patient had at first a less positive response. The concentration of oxalate (mg/L) decreased with 36%. The 24 hours oxaluria in the urine decreased by 55% however not yet reaching the end points. After 1 year and 7 months (and several periods of less compliance to the medication), the patient had urinary values (n=3) of oxalate of 28 mg/L, which was clearly below the oxaluria ULN.

All patients were able to reach the three end points:
oxaluria of less than 0.50 mmol/L (44 mg/L) associated with calciuria below 4.0 mmol/L (160 mg/L) (Table 2, and FIG. 4A, 4B, 4E)
a stable and normal phosphate concentrations in serum (0.9-1.5 mmol/L; 30-45 mg/L) and urine (>350 mg/L) (Table 2, and FIG. 4D)
no increasing concentrations of the calciuria, remaining stable over the treatment period (Table 2, and FIG. 4C).

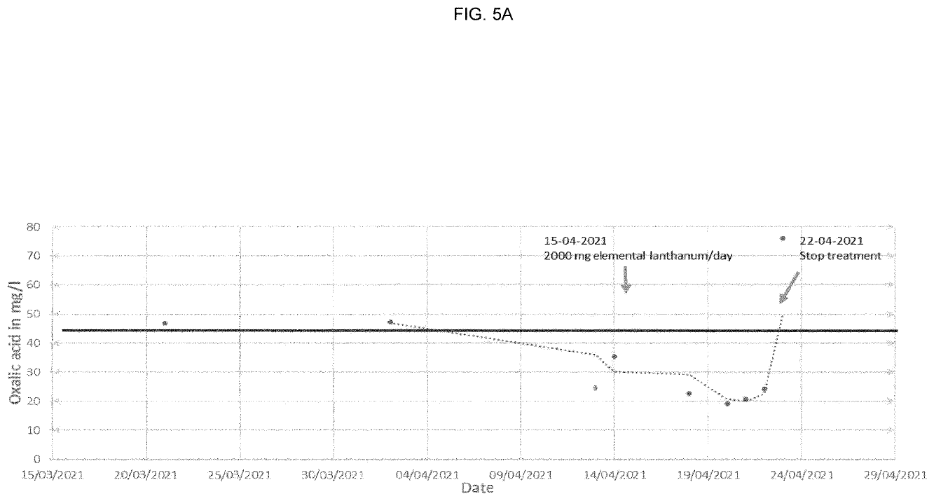

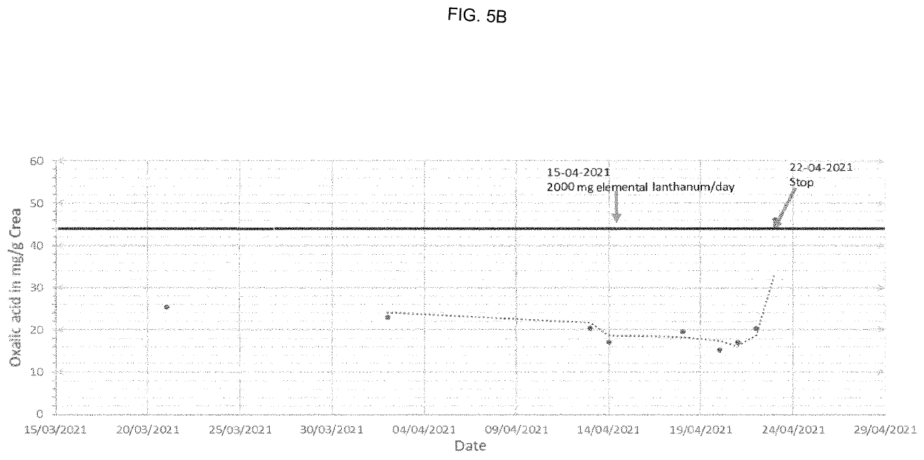

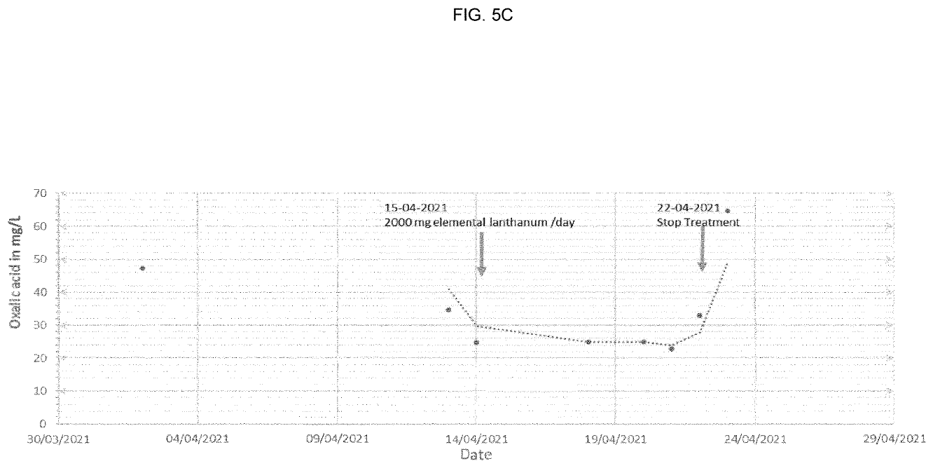

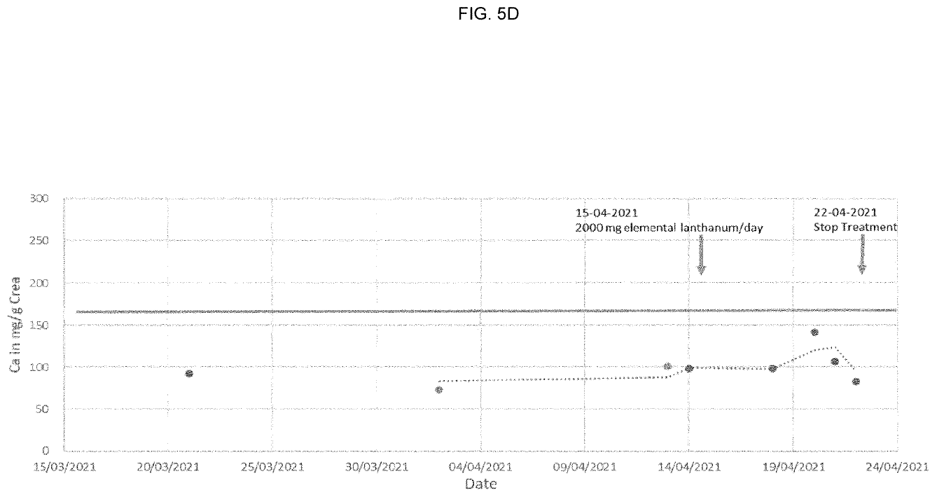

The results of the secondar hyperoxaluria patient are given in FIG. 5. FIG. 5 shows: (A) oxaluria (mg/L), (B) oxaluria (mg/gr creatinine), (C) serum oxalate (mg/L), (D) calciuria (mg/gr creatinine), and (E) phosphaturia (mg/gr creatinine) before, during, and after treatment with the pharmaceutical dosage form according to an embodiment of the invention in a patient with secondary hyperoxaluria (Patient 5, idiopathic calcium oxalate urolithiasis type).

The secondary hyperoxaluria patient responded by decreasing his oxaluria from 49 mg/L to 21 mg/L (i.e., decrease by 58%) already after one week of treatment with the pharmaceutical dosage form illustrating the invention given in 2 doses of 1000 mg elemental lanthanum each (i.e., 8×250 mg=2000 mg elemental lanthanum/day) (FIG. 5). One dose of 4 times 250 mg elemental lanthanum was given in the morning and one dose of 4 times 250 mg elemental lanthanum was given in the evening. After arrest of the treatment, the oxalate level increased to the pre-treatment levels after two days. The rather quick normalization of the different oxaluria parameters in the secondary hyperoxaluria patient can be explained by the lower oxalate pool present in the patient compared to primary oxaluria patients.

Patients did not report any side effect during the treatment period.

### Discussion

The present pharmaceutical dosage form illustrating the invention comprising a well-defined amount of lanthanum carbonate allows the targeted release of lanthanum at a precise location, i.e.

in the distant part of the gastrointestinal tract, e.g. the ileum, cecum, and early colon, whereby all physiological parameters are maximally fulfilled. After testing several formulae, treatment with the pharmaceutical dosage form according to an embodiment of the invention, particularly capsules comprising four layers of coating configured for targeted delivery to the distal part of the gastrointestinal tract of a subject, resulted in:
release of lanthanum carbonate in the late small intestine (ileum) and early colon, as tested using the CT scan technique over 6 h. after ingestion of the pharmaceutical dosage form according to an embodiment of the invention.
optimal gastrointestinal chelation of oxalate (found by the present inventors to be absorbed mainly in the ileum and colon) resulting in substantial decrease in its urinary concentration below 0.50 mmol/L (44 mg/L). Hence, the administration of the pharmaceutical dosage form according to an embodiment of the invention resulted in treatment of the primary or secondary hyperoxaluria.
the unchanged, stable, and normal urinary phosphate concentrations in patients with primary hyperoxaluria and secondary hyperoxaluria resulting in a normal phosphate balance during the whole treatment period. These observations indicate the more proximal gastrointestinal reabsorption of phosphate.
the absence of an increase in calciuria in patients treated with the pharmaceutical dosage form according to an embodiment of the invention, in contrast with the increased calciuria observed with all other current powerful phosphate binders (such as aluminum, Sevelamer, calcium carbonate) and which may contribute to promote calcium oxalate crystal formation.
the pharmaceutical dosage form according to an embodiment of the invention maintains or even slightly decreases calcium in the urine during the whole treatment period. This stable, normal, or in some cases even slightly decreasing calciuria may be due to the calcium channel blocking effect of lanthanum in the distal part of the GI tract and may contribute to arrest the calcium oxalate crystal formation.
the quick normalization of the different oxaluria parameters in the secondary hyperoxaluria patient can be explained by the lower accumulated oxalate pool present in these patients compared to the primary oxaluria patients.

The end result of treatment with the pharmaceutical dosage form according to an embodiment of the invention is unique. This is the first time that a therapy using stops/arrests the calcium oxalate crystal formation (below 44 mg/L or 0.50 mmol/L oxaluria associated with a calciuria below 160 mg/L or 4.0 mmol/L) in the tubular fluid of the kidney/urine.

Advantageously, the pharmaceutical dosage form according to an embodiment of the invention is not limited to treatment of one kind of primary hyperoxaluria or secondary hyperoxaluria (in contrast to e.g. Lumasiran (Oxlumo, Alnylam) which inhibitory activity of hepatic oxalate synthesis is limited to type 1 primary hyperoxaluria) but is active in any form of primary hyperoxaluria since the pharmacological activity consists in an optimal chelation of oxalate ingested and secreted in the distal parts of the gastrointestinal tract (as shown in Table 2).

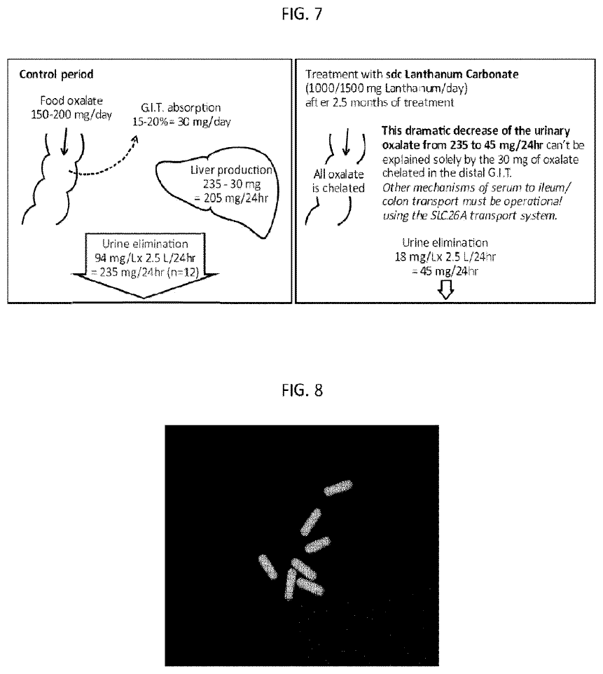

In addition, treatment with a pharmaceutical dosage form according to an embodiment of the invention (herein also referred to as “special designed capsule lanthanum carbonate” or “sdc Lanthanum Carbonate” or “sdcLC”) involves more than chelation of the daily dietary oxalate. Without being bound to theory, it is hypothesized that due to treatment with the pharmaceutical dosage form according to an embodiment of the invention oxalate is secreted from the serum to the colon, potentially mediated by SLG26A transporters. As can be seen in FIG. 4A, treatment of patient 1 with a pharmaceutical dosage form according to an embodiment of the invention for 2.5 months resulted in a concentration of oxalate in the urine below the upper limit of normal (ULN: 44 mg/L). Patient 1, having PH type 1 and normal renal function, had a mean oxaluria of 94 mg/L±4.8 mg/L (control values n=12, 24 hr urine collections) during the control period (FIG. 4A, Table 2) and a mean daily diuresis of 2.5 L/day±0.3 L/day. Given an average food oxalate concentration of 150-200 mg/day (i.e., about 2 mmol/day) and 15-20% is absorption in the gut (Holmes et al., 2000, Kidn Intern, 57, 1662-1667), the daily food oxalate load is approximately 30 mg/day (i.e., 200 mg/day×15%) (FIG. 7).

After 6-8 weeks of treatment, the urinary oxaluria decreased from 94 mg/L to 18 mg/L (FIG. 4A, Table 2). During treatment with a pharmaceutical dosage form according to an embodiment of the invention one may accept that almost all oxalate in the food was chelated in the distal GI tract by the lanthanum, since 1500 mg Lanthanum (11 mmol) was given per day, representing a molar lanthanum/oxalate ratio of 5.5 (i.e., 11 mmol/2 mmol). Hence, the food oxalate was no more available for body oxalate loading.

The chelation of 30 mg/day of food oxalate was insufficient to explain pronounced decrease in the daily urinary elimination of oxalate over time of 235 mg (i.e., 94 mg/L×2.5 L) to 45 mg (i.e., 18 mg/L×2.5 L). The liver production of oxalate was still intact and maintained, and there are no systemic effects of lanthanum. Further, the gastro-intestinal absorption of lanthanum is negligeable (0.01%) (Persy et al., 2006, Sem Dialysis, 19, 198-199).

The above thus suggests that another mechanisms in addition to chelation of oxalate from the food was operational in the oxalate balance in case of treatment of primary hyperoxaluria patients with a pharmaceutical dosage form according to an embodiment of the invention. One of them, most likely, is oxalate redirection from the serum to the distal intestinal lumen by secretion of oxalate (mediated by the SLC26A6 transporter) further depleting the oxalate from the body. This secreted oxalate was immediately chelated in the distal part of the GI tract, i.e. ileum, cecum and/or colon, containing free lanthanum released from the pharmaceutical dosage form (FIG. 7). The chelation products advantageously were eliminated through the stools.

### Conclusion

The overall effect of the pharmaceutical dosage form according to an embodiment of the invention consists in an optimal chelation of the daily ingested oxalate in the distal part of GI tract, stimulating the oxalate redirection from the serum to the intestinal lumen by secretion of oxalate (e.g. mediated by the SLC26A6 transporter).

Optimal chelation of oxalate resulting in the substantial decrease of urinary oxalate combined with a calcium channel blocking effect of the pharmaceutical dosage form according to an embodiment of the invention in the gut maintaining or slightly decreasing the urinary calcium concentrations arrests the calcium oxalate crystal formation. In addition, the pharmaceutical dosage form according to an embodiment of the invention leads to treatment of hyperoxaluria and a normal and stable phosphate concentration in the urine indicating absence of progressive phosphate depletion in any type of primary and in secondary hyperoxaluria.

### Example 4: Investigation of Localisation and Release of a Pharmaceutical Dosage Form Illustrating the Invention during Gastrointestinal Tract Passage Using Sequential X-Ray Radiographer and Ultra-Low-Dose Computed Tomography

The aim of the investigation is the validation of the concept that a pharmaceutical dosage form illustrating the invention allows the release of lanthanum in the ileum and colon. The pharmaceutical dosage form illustrating the invention (herein also referred to as “special designed capsule lanthanum carbonate” or “sdc Lanthanum Carbonate” or “sdcLC”) is prepared as described in Example 1, i.e., a capsule comprising 250 mg elemental lanthanum, or in the same way but the capsule comprising up to 500 mg elemental lanthanum. The capsules are seen as radiopaque structures on X-ray imaging as illustrated in FIG. 8.

### Task Validation Study Design and Execution

### Preliminary Results

Lanthanum is a metallic chemical element with the atomic mass of 138.91 g/mol and therefore is radiopaque and easily visible on X-ray images. In order to achieve the correct pharmacological activity, the present inventors found that lanthanum has to arrive intact into the ileum, cecum, and/or colon and at the same time not interfere with the phosphate reabsorption in the proximal part of the gastrointestinal tract. A trial was conducted using a Shimadzu Sonialvision G4 radiographer to perform a small intestinal transit study (see Example 2).

### Study Design

Out of this preliminary study, a prospective study is performed using a serial ultra-low dose computed tomography (ULDCT) as replacement modality for sequential RX imaging, thus optimizing topography and to be able to show in detail where the lanthanum dissolves. The radiation dose may be optimized so that it can be lower than the dose given in a classic small intestine transit study. The ULDCT scan protocol may be derived by adjusting the standard CT abdomen protocol with reduced exposure parameters and the use of iterative reconstruction available on the system, Adaptive Statistical Iterative Reconstructions (ASIR®). Tube voltage is reduced from 120 kV to 100 kV and the noise index increased from 28 to 70, with a tube current window between 30 mA and 400 mA to maintain a constant image quality. The data are reconstructed with 100% ASIR. The effective dose of one ULDCT scan of the abdomen (CTDIvol=1.7 mGy, DLP=58 mGycm) is between 0.3-0.4 mSv, compared to 10.4 mSv for a standard diagnostic CT-scan (CTDIvol=12.7 mGy and DLP=673 mGycm). For a standard abdominal plain radiograph the effective dose is about 0.8 mSv (2 projections).

Every patient is to be expected to have a somewhat different pace of intestinal activity and therefore variable distal movement of the lanthanum particles. In order to localize the release of lanthanum of the capsules, sequential scans using following time interval are taken: immediately after ingestion of three capsules (doses are different for children of age 6-11 year and adults, and will be determined for the children based on body surface) and every 30 minutes until all lanthanum particles, visible as diffuse punctiform intra-intestinal radiopaque dots, are located in the colon. We expect that this takes no longer than 360 minutes (6 hours).

A number of normal volunteers (n=5) and patients with PH (n=5, 3 adults and 2 children between 6-11 years of age) participate in this study. They are administered pharmaceutical dosage forms according to embodiments of the invention (e.g., 3 capsules of 500 mg elemental lanthanum in adults and 3 small capsules of 250 mg elemental lanthanum in children) and ULDCT is performed before the start of the actual treatment.

The GI tract is divided in 6 compartments: stomach, duodenum, jejunum, ileum, early colon, late colon. The presence of intact versus released lanthanum is scored in a blinded manner during the analysis of the pictures of normal volunteers versus PH patients. Scoring is 0: if no lanthanum particles are present in the compartment, 1: if a complete capsule is present, 2: if large fragments of the capsule are present, 3: if larger and punctiform fragments are visible and 4: if only punctiform fragments are present in a compartment.

### Deliverables

Clinical validation of the localization of the release of lanthanum carbonate from the capsules and final concentration of lanthanum in ileum and colon.

### Milestones

This study is organized over a period of 1.5 year in order to have the time to recruit the normal volunteers and the patients before the start of the treatment with sdcLC.

### Risks

Scientific risks: Validation study fails to meet success criteria: score low in view of results obtained in the proof of concept performed three times in a normal individual.

Slow recruitment of normal individuals and patients in this study, the fact not to find the number of adults and children to participate: score low. Until now, we have not been confronted with a refusal of patients with PH. The extra effort of 24 hr collection of urine is never refused. Participating a half day for flow cytometric analysis of all the urine samples, and staying in the lab is accepted. Correct and open information to the patients and relevance of the investigation is helpful.

The technique proposed results in an ultra-low dose CT (ULDCT) of radiation.

### Example 5: Clinical Validation Study of the Pharmacological Efficacy of a Pharmaceutical Dosage Form According to an Embodiment of the Invention in Patients with Primary Hyperoxaluria

A controlled, randomized multicenter, international (Belgium, Algeria, Tunisia, Morocco), double blinded, cross-over clinical trial in 30 primary hyperoxaluria (PH) patients is organized. After 1 month of control, a treatment period of 2 times 6 months with a wash-out period of 2 months is applied. Randomization is organized centrally and blinding is applicable at the treatment sites, for the patients as well as for the treating physicians. Treatment will start for instance with about 1000 mg/day of elemental lanthanum in a pharmaceutical dosage form according to an embodiment of the invention, namely sdc Lanthanum Carbonate capsules prepared as described in Example 1.Treatment may be gradually increased for instance to about 2000 mg/day of elemental lanthanum. For children of 5-15 years of age a dose adapted to the body surface will be prescribed in consult with the responsible paediatrician. In a random selection of patients in the placebo group, investigated medicinal product (IMP) is also gradually increased accordingly. Results of the treatment are investigated using 24hour collections of urine at a regular base (2 times per month) for the central determination of oxalate. The hypothesis tested are: (1) whether treatment with sdcLC during 6 months reduces oxaluria to a level where calcium oxalate crystals no longer form in the urine (ULN: 44 mg/L), hence reduces oxaluria to at most 44 mg/L, (2) whether treatment with sdcLC during 6 months reduces oxaluria with 60% in at least 75% of the patients.

### Example 6: Additional Applications for Treatment of Patients Using a Pharmaceutical Dosage Form According to an Embodiment of the Invention

### Clinical Validation of the Additive Effect of sdcLC Given to Patients Treated with Lumasiran and Presenting a Suboptimal Result with Lumasiran Alone

The results of the lumasiran study (Garrelfs et al., 2021, N Engl J Med, 384(13): 1216-1226) indicate that the percentage of patients with PH having a 24-hr urinary oxalate excretion below the upper limit of the normal (ULN) at month 6 is 52% (ULN=0.51 mmol/L or 44 mg/L). Hence some patients treated with this medication do not reach an urinary oxalate level which is very low or crystal formation free. Patients are considered for recruitment after treatment with lumasiran for 6 months according to the correct EMA lumasiran treatment schedule (i.e., starting with 4 doses with an interval of 1 month followed by 1 dose every 3 months in adults). If after 6 months, the primary end point for lumasiran treatment is not reached, the patient is asked to participate in this observational study. The primary end point for lumasiran treatment is reaching a 24-hour urinary oxalate excretion lower than about 1.5 times the upper limit of the normal (ULN), namely 68 mg/24 hrs. Patients (n=5) are additionally treated for instance with sdcLC of about 1000 mg/day of elemental lanthanum. Treatment may be gradually increased to about 2000 mg/day of elemental lanthanum. Sampling is identical to the requirements described in the clinical validation study of Example 5.

A second group of PH patients is also a candidate for combined treatment of lumasiran and sdcLC, namely PH patients with an end stage renal failure in dialysis and in need of a renal transplantation. In order to decrease substantially the oxalate load present in the body of this type of patients a powerful intense clearing treatment is needed. This could consist in the combination of lumasiran treatment and sdcLC in combination with frequent dialysis over a period of at least 2-4 months. Following the plasma levels of oxalate is mandatory because the patients have no significant renal function. sdcLC treatment and sampling is identical to the requirements described in the clinical validation study of Example 5.

### Clinical Validation of the Effect of sdcLC Given to Patients Having Primary or Secondary Hyperoxaluria and Impaired Renal Function

Patients with primary hyperoxaluria and chronic kidney disease (CKD3-5), thus showing significant accumulation of calcium oxalate in their tissues and who should receive treatment to potentially solubilize and remove most of the calcium oxalate through secretion from the serum to the ileum and colon are also candidate for treatment with sdcLC capsules.

Further, patients with PH and terminal CKD or impaired renal function (eGFR<40 ml/min) who are on dialysis and in need of kidney and liver transplant are also candidate for treatment with sdcLC capsules. Since a few years, doctors have tried to limit transplantation to a kidney transplant since lumasiran can largely suppress liver production of oxalate. Since lumasiran does not always work satisfactorily and the serum levels may remain too high to safely perform the kidney transplantation, sdcLC treatment with its high capacity of chelation of secreted oxalate and elimination via the intestine is highly interesting. For the avoidance of doubt, the kidney to be transplanted must be placed in an environment of oxalate concentrations below 44 mg/L in the urine in order to prevent crystal formation after the kidney transplant when urine formation starts again.

A further group of patients to be treated with sdcLC are patients with secondary hyperoxaluria with moderate renal insufficiency.

### Comparative Example: Administration of Uncoated Lanthanum Carbonate to a Human Subject

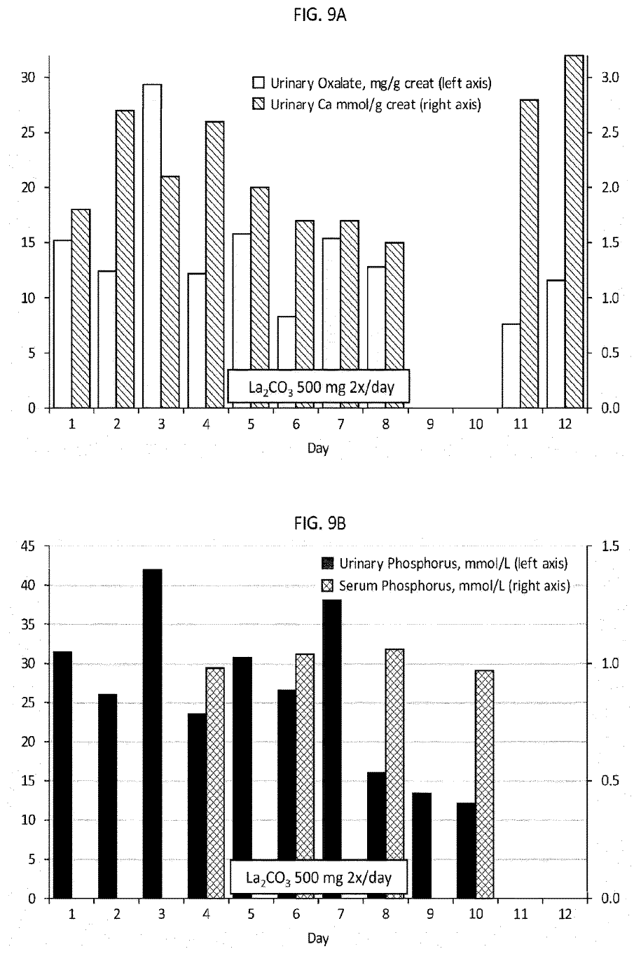

In order to compare the effect of uncoated lanthanum carbonate versus coated lanthanum carbonate capsules illustrating the invention (see Example 1 and FIG. 3) on the oxaluria, calciuria, and phosphaturia, a human normal volunteer was subjected to oral administration of Fosrenol (Shire Pharmaceuticals), i.e., lanthanum carbonate as such, in a dosage of 500 mg elemental lanthanum/day (2×250 mg elemental lanthanum). The normal volunteer (male, 74 years of age, eGFR 73 ml/min/1.73 m2) had healthy kidney function, no diabetes, normal blood pressure and no other pathologies. FIG. 9 represents graphs illustrating (A) the urinary oxalate concentration (mg/g creatinine, dark grey bars) and the urinary calcium (Ca) concentration (mmol/g creatinine, light grey bars), or (B) urinary phosphate (P) concentration (mmol/L, grey bars) and serum phosphate (P) concentration (mmol/L, black bars) of the normal volunteer after oral ingestion of Fosrenol. The figures show that during this short clinical test, administration of uncoated lanthanum carbonate caused a decrease of urinary phosphate concentration (FIG. 9B) while no effect was seen on serum phosphate concentration (FIG. 9B) or urinary oxalate concentration (FIG. 9A). These results are in contrast with the results obtained after administration of the pharmaceutical dosage form illustrating the invention (Example 1, FIG. 3) which showed a decrease of more than 40% decrease in urinary oxalate compared to the control values (see FIG. 3). No clear effect was seen on the phosphaturia and calciuria (see FIG. 3). The administration of uncoated lanthanum carbonate decreased urinary phosphate and had almost no effect on oxaluria, suggesting preferred chelation of phosphate over oxalate (phosphate/oxalate ratio in food is 10/1), thereby confirming that lanthanum carbonate given to subjects with intact or relative preserved renal function (eGFR>40 ml/min/1.73 m²) will result in phosphate depletion with serious complications over time, such as osteomalacia.

1. An orally administrable pharmaceutical dosage form comprising lanthanum or a pharmaceutically acceptable salt or oxide thereof, wherein the pharmaceutical dosage form is configured for targeted delivery of the lanthanum or pharmaceutically acceptable salt or oxide thereof to one or more of the ileum, cecum, or colon of a human subject, and wherein the dosage form comprises a polymer which does not disintegrate at a pH of below 6.8.

2. The pharmaceutical dosage form of claim 1, wherein the pharmaceutical dosage form is configured for targeted delivery of the lanthanum or pharmaceutically acceptable salt or oxide thereof to one or more of the ileum, cecum, or ascending colon of the human subject.

3. The pharmaceutical dosage form of claim 1, wherein the pharmaceutical dosage form is a solid or semisolid dosage form.

4. The pharmaceutical dosage form of claim 1, wherein the pharmaceutical dosage form is a capsule or a tablet.

5. The pharmaceutical dosage form of claim 1, wherein the pharmaceutical dosage form comprises a coating configured for targeted delivery of the lanthanum or pharmaceutically acceptable salt or oxide thereof to one or more of the ileum, cecum, or colon of a human subject, wherein the coating comprises a polymer which does not disintegrate at a pH of below 6.8.

6. The pharmaceutical dosage form of claim 1, wherein the pharmaceutical dosage form comprises a copolymer of methacrylic acid and C₁₋₄alkylester of methacrylic acid.

7. The pharmaceutical dosage form of claim 4, wherein the capsule is made of one or more materials selected from the group consisting of cellulose acetate phthalate (CAP), hydroxypropyl methylcellulose (HPMC), gelatin, pullulan, starch, a water-soluble polyvinyl derivative, a polyethylene glycol, and a copolymer of C₁₋₄alkylester of (meth)acrylic acid and (meth)acrylic acid.

8. The pharmaceutical dosage form of claim 1, wherein the pharmaceutical dosage form comprises at least three layers of a coating.

9. The pharmaceutical dosage form of claim 1, wherein the pharmaceutically acceptable salt or oxide of lanthanum is lanthanum carbonate or lanthanum oxide.

10. The pharmaceutical dosage of claim 1, wherein the pharmaceutical dosage form comprises 100 mg to 4000 mg of elemental lanthanum.

11. The pharmaceutical dosage form of claim 1, wherein the pharmaceutical dosage form is a capsule filled with a composition comprising the lanthanum or pharmaceutically acceptable salt or oxide thereof, and coated with a copolymer of methacrylic acid and C₁₋₄alkylester of methacrylic acid.

12. The pharmaceutical dosage form of claim 1, wherein the pharmaceutical dosage form is a capsule filled with a composition comprising the lanthanum or pharmaceutically acceptable salt or oxide thereof, and coated with a copolymer of methacrylic acid and C₁₋₄alkylester of methacrylic acid, wherein the capsule comprises at least four layers of the coating.

13. The pharmaceutical dosage form of claim 1, wherein the pharmaceutical dosage form is a capsule filled with a composition comprising the lanthanum or pharmaceutically acceptable salt or oxide thereof, and coated with a copolymer of methacrylic acid and C₁₋₄alkylester of methacrylic acid, wherein the thickness of the coating is at least 20.0 μm.

14. (canceled)

15. (canceled)

16. (canceled)

17. A method of treating one or more of hyperoxaluria, nephrolithiasis, nephrocalcinosis, or oxalosis in a human subject in need of such a treatment, the method comprising administering a therapeutically effective amount of a pharmaceutical dosage form of claim 1 to the subject.

18. The method according to claim 17, wherein the hyperoxaluria is primary hyperoxaluria or secondary hyperoxaluria.

19. The method according to claim 17, wherein the pharmaceutical dosage form is administered:
once or twice a day;
in an amount of from 100 mg to 2000 mg lanthanum per administration;
in an amount of from 100 mg to 4000 mg lanthanum per day; and/or
for a period of at least one month; such as for a period of at least two months.

20. The pharmaceutical dosage form of claim 4, wherein the capsule is filled with a composition comprising the lanthanum or pharmaceutically acceptable salt or oxide thereof; preferably wherein the lanthanum or the pharmaceutically acceptable salt or oxide thereof is in powder form.

22. The pharmaceutical dosage form of claim 6, wherein the copolymer is a copolymer of methacrylic acid and methyl methacrylate.

22. The pharmaceutical dosage form of claim 61 wherein the pharmaceutical dosage form comprises lanthanum carbonate.

23. The pharmaceutical dosage form of claim 8, wherein the pharmaceutical dosage form comprises at least four layers of the coating; and/or wherein the thickness of the coating is at least 20.0 μm, at least 25.0 μm, at least 30.0 μm, or at least 35.0 μm.
